(19) United States
(12) Patent Application Publication
(10) Pub. No.: US 2025/0332129 A1
(43) Pub. Date: Oct. 30, 2025

(54) LIPOPEPTIDE COMPOUND AND TREATMENT OF PAIN DISORDER

(71) Applicants: INSTITUT NATIONAL DE LA SANTE ET DE LA RECHERCHE MEDICALE, Paris (FR); UNIVERSITE TOULOUSE III – PAUL SABATIER, TOULOUSE (FR); CENTRE NATIONAL DE LA RECHERCHE SCIENTIFIQUE, Paris (FR); CENTRE HOSPITALIER UNIVERSITAIRE DE TOULOUSE, TOULOUSE (FR); INSTITUT NATIONAL DE RECHERCHE POUR L'AGRICULTURE, L'ALIMENTATION ET L'ENVIRONNEMENT, Paris (FR); ECOLE NATIONALE VÉTÉRINAIRE DE TOULOUSE, TOULOUSE (FR); ECOLE NATIONALE SUPERIEURE DE CHIMIE DE MONTPELLIER, MONTPELLIER CEDEX 5 (FR); UNIVERSITE DE MONTPELLIER, MONTPELLIER (FR)

(72) Inventors: Nicolas CENAC, TOULOUSE CEDEX 3 (FR); Justine BERTRAND-MICHEL, TOULOUSE CEDEX 4 (FR); Thierry DURAND, MONTPELLIER CEDEX 5 (FR); Jean-Marie GALANO, MONTPELLIER CEDEX 5 (FR); Amandine HUEBER, TOULOUSE CEDEX 3 (FR); Pauline LE FAOUDER, TOULOUSE CEDEX 4 (FR); Alexandre GUY, MONTPELLIER CEDEX 5 (FR); Sarah MAUREL, TOULOUSE CEDEX 3 (FR)

(21) Appl. No.: 18/855,427

(22) PCT Filed: Apr. 14, 2023
(86) PCT No.: PCT/EP2023/059771

§ 371 Date: Oct. 9, 2024

Related U.S. Application Data
(63) Continuation of application No. 17/721,421, filed on Apr. 15, 2022, now abandoned.

(30) Foreign Application Priority Data
Sep. 23, 2022 (EP) 22306408.0

(51) Int. Cl. A61K 31/197; A61P 29/00

(52) U.S. Cl. CPC A61K 31/197; A61P 29/00

(57) ABSTRACT

The invention is based on the discovery of a novel compound with analgesic properties which could be used as a new tool for the treatment of pain disorders such as visceral pain. Thus, the invention relates to novel lipopeptide compound, derived from gamma-aminobutyric acid. The invention also relates to a lipopeptide compound according to the invention for the treatment of treating pain disorder, such as somatic pain and visceral pain.

### FIELD OF THE INVENTION

The invention relates to a lipopeptide compound and their use for treating pain disorders. In particular, the invention concerns novel lipopeptide compounds and their uses for treating visceral pain and somatic pain.

### BACKGROUND OF THE INVENTION

Pain is an unpleasant feeling often caused by intense or damaging stimuli. The International Association for the Study of Pain's widely used definition states: “Pain is an unpleasant sensory and emotional experience associated with actual or potential tissue damage, or described in terms of such damage”. For example, chronic pain is a common problem that constitutes a major challenge to healthcare providers because of its complex natural history, unclear etiology, and poor response to therapy. Chronic pain is a poorly defined condition. Most authors consider ongoing pain lasting longer than 6 months as diagnostic, whereas others have used 3 months as the minimum criterion. In chronic pain, the duration parameter is used arbitrarily. Some authors suggest that any pain that persists longer than the reasonable expected healing time for the involved tissues should be considered chronic pain. The pathophysiology of chronic pain is multifactorial and complex and is still poorly understood. Various neuromuscular, reproductive, gastrointestinal, and urologic disorders may cause or contribute to chronic pain.

Irritable bowel syndrome (IBS) is a functional gastrointestinal (GI) disorder characterized by recurrent episodes of abdominal pain/discomfort and bowel habit changes (e.g. constipation, diarrhea). With a global prevalence of ˜11%, IBS constitutes one of the most common conditions leading to gastroenterological referral, and results in a considerable disease burden. While the pathophysiology of IBS is not fully understood, visceral hypersensitivity (VH; enhanced sensitivity of the intestinal wall to local stimuli) has been proposed as a key mechanism underlying abdominal pain, one of the most debilitating and most troublesome symptoms of this disorder. Current treatments for IBS are mainly symptoms orientated; however, the overall efficacy is low and there are no drugs specifically approved for abdominal pain. Thus, selective pharmacological tools targeting VH may be considered a suitable therapeutic approach for visceral pain treatment and development of novel IBS therapies.

Data from clinical research suggest that certain probiotic bacterial strains have the potential to modulate abdominal pain in IBS. Nonetheless, this data differs considerably among studies due to the probiotic bacterial strains used for the treatment and the heterogeneity of IBS groups included. Moreover, the mechanisms of action responsible for the claimed therapeutic effects differ from one strain to another.

WO 2018/197666 A1 describes the isolation and structural elucidation of new metabolites encoded by the pks island of Escherichia coli Nissle 1917 (EcN), the active component of Mutaflor® (Ardeypharm GmbH, Herdecke, Germany), a probiotic drug licensed in several countries for the treatment of multiple intestinal disorders. These compounds, in particular C12-Asn-GABA, shown potent anti-nociceptive properties in vitro and in vivo. While an increased number of small molecules derived from the colibactin encoding hybrid PKS-NRPS biosynthetic gene clusters have been described, this was the first study characterizing a non-genotoxic bioactive metabolites in vivo. This analgesic compound produced by probiotic bacteria may represent a promising therapeutic agent in somatic pain and visceral pain.

However, it remains essential to rapidly develop novel analgesic molecules as drug candidates for treating somatic pain and visceral pain.

### SUMMARY OF THE INVENTION

An objective of the present invention is to provide new compounds which can be used in the treatment of somatic pain and visceral pain.

The present invention is based on a study investigating the relationship between prenatal stress (PS), gut microbiota and visceral hypersensitivity with a focus on bacterial lipopeptides containing GABA.

For that study, the inventors have developed a model of PS in mice and evaluated, in adult offspring, visceral hypersensitivity to colorectal distension, colon inflammation, barrier function and gut microbiota taxonomy. Then, they quantified the production of lipopeptides containing GABA by mass spectrometry in a specific strain of bacteria decreased in PS, in PS mouse colons and in feces of IBS patients and healthy volunteers. Finally, the effect of these lipopeptides on PS-induced visceral hypersensitivity was assessed. In parallel, the GABA containing lipopeptide content of feces of IBS human patients was compared to that of healthy volunteers.

The results of that study shows that prenatally stressed mice of both sexes presented visceral hypersensitivity, no overt colon inflammation or barrier dysfunction but a gut microbiota dysbiosis. The dysbiosis was distinguished by a decreased abundance of Ligilactobacillus murinus, in both sexes, inversely correlated with visceral hypersensitivity to colorectal distension in mice. An isolate from this bacterial species produced several lipopeptides containing GABA. Further, it was shown that a lipopeptide C16LeuGABA, which was found in human feces, was decreased in feces of IBS patients compared to healthy volunteers. C16LeuGABA was then shown to have a potent inhibitory activity on sensory neurons activation, in particular a more potent activity than other anti-nociceptive lipopeptides containing GABA.

Thus, the invention relates to a lipopeptide compound derived from gamma-aminobutyric acid of formula I (see below).

The invention also relates to a lipopeptide compound according to the invention for use in the treatment of a pain disorder, such as somatic pain or visceral pain.

A further object of the invention relates to a therapeutic composition comprising lipopeptide compound.

Finally, a method to synthesize the lipopeptide compound is also described.

### DETAILED DESCRIPTION OF THE INVENTION

### Lipopeptide Compound

In a first aspect, the present invention provides a compound having the following structure of formula (I):

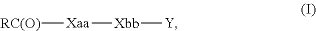

wherein
   R is a C5-C19 (fatty) hydrocarbon chain,
   Xaa is any amino acid such as leucine, phenylalanine, isoleucine, or alanine,
   Xbb is a gamma-aminobutyric acid moiety,
   Y is —OH or NH₂,
or a pharmaceutical acceptable salt thereof.

In an embodiment, the compound according to the present invention are of Formula (I):

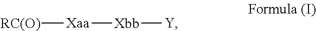

wherein
   R is a C5-C19 linear or branched hydrocarbon chain selected from the group consisting of alkyl, alkene, and alkyne,
   Xaa is leucine, phenylalanine, isoleucine, or alanine,
   Xbb is HNCH₂CH₂CH₂CO,
   Y is —OH or NH₂,
and wherein Xbb is linked to Xaa through its amine functional group and wherein the RC(O) group is at the N terminal side and Y is a C terminal side,
or a pharmaceutical acceptable salt thereof.

Structurally, cell membranes constitute a complex set of lipids, proteins and sugars (or oses) organized on the basis of a double phospholipid sheet. Thus, cell membranes act as real physicochemical barriers through the regulation of the cellular matter exchange. For instance, GABA alone is not able to cross cell membrane. The ability of the compound of the invention to cross the cellular epithelial barrier is linked to the (fatty) hydrocarbon chain of the compound which must contain at least 5 carbons to cross membrane cells.

It will be appreciated that in the present invention, unless the context clearly dictates otherwise, referring to “the compound of the present invention” is equivalent to referring the “the compound” “lipopeptide compound” or “gamma-aminobutyric acid compound” or “inhibitor”.

The term “C5-C19 (fatty) hydrocarbon chain” group means a saturated or unsatured hydrocarbon chain, linear or branched, comprising from 5 to 19 carbon atoms, such as for example an alkyl, alkene or alkyne, etc.

In one embodiment, R is selected from the list consisting of alkyl, alkene or alkyne.

In a specific embodiment, R is a C5-C19 alkyl. In a more specific embodiment R is a C11 alkyl.

“C5-C19 alkyl” group means a saturated hydrocarbon chain, linear or branched, comprising from 1 to 19 carbon atoms.

In a specific embodiment, C5-C19 alkyl of the compound according to the present invention has the following structure:

CH3-CyHx-, where y=4 to 18, x=2y. In a specific embodiment, y=8 to 14. For example, y=10.

In an embodiment, R is a C12 alkyl or a C14 alkyl or a C16 alkyl.

In an embodiment, R is a C16 alkyl.

The term gamma-Aminobutyric acid (γ-Aminobutyric acid) (also called GABA) means the chief inhibitory neurotransmitter in the mammalian central nervous system. It plays the principal role in reducing neuronal excitability throughout the nervous system (see Watanabe M, et al (2002). “GABA and GABA receptors in the central nervous system and other organs”. Int. Rev. Cytol. 213. p. 1-47). Although in chemical terms it is an amino acid (as it has both a primary amine and a carboxylic acid functional group), GABA is rarely referred to as such in the scientific or medical community. By convention the term “amino acid”, when used without a qualifier, refers specifically to an alpha amino acid. GABA is not an alpha amino acid, meaning the amino group is not attached to the alpha carbon so it is not incorporated into proteins.

Gamma-Aminobutyric acid Formula is

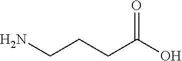

The term “Gamma-aminobutyric acid moiety” means a moiety of Formula HNCH₂CH₂CH₂CO. “HNCH₂CH₂CH₂CO” can interchangeably be represented by “—HNCH₂CH₂CH₂CO—” or “—NHCH₂CH₂CH₂CO—”.

In an embodiment, Xbb is HNCH₂CH₂CH₂CO.

The GABA moiety is preferably linked to Xaa amino acid through its amine functional group.

In some embodiments, the GABA moiety is preferably linked to any amino acid such as leucine, phenylalanine, isoleucine, or alanine through its amine functional group.

In a specific embodiment, Y is OH—.

In another embodiment Xaa is any amino acid as described herein.

In another embodiment Xaa is leucine, phenylalanine, isoleucine, or alanine.

In another embodiment, Xaa is leucine or isoleucine, preferably is leucine.

In another embodiment Xaa is leucine, phenylalanine, isoleucine, or alanine or an equivalent hydrophobic or non-polar amino acid selected from the list valine, glycine, proline or methionine.

In a preferred embodiment, for the compound of formula (I), the RC(O) group is at the N terminal side of the lipopeptide, and the GABA moiety is a C terminal side of the lipopeptide.

As used herein, the term “amino acid” refers to natural or unnatural amino acids in their D and L stereoisomers for chiral amino acids. It is understood to refer to both amino acids and the corresponding amino acid residues, such as are present, for example, in peptidyl structure.

Natural and unnatural amino acids are well known in the art. Common natural amino acids include, without limitation, alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), tryptophan (Trp), tyrosine (Tyr), and valine (Val). Uncommon and unnatural amino acids include, without limitation, allyl glycine (AllylGly), norleucine, norvaline (Avl), biphenylalanine (Bip), citrulline (Cit), 4-guanidinophenylalanine (Phe(Gu)), homoarginine (hArg), homolysine (hLys), 2-naphtylalanine (2-Nal), ornithine (Orn) and pentafluorophenylalanine.

Amino acids are typically classified in one or more categories, including polar, hydrophobic, acidic, basic and aromatic, according to their side chains. Examples of polar amino acids include those having side chain functional groups such as hydroxyl, sulfhydryl, and amide, as well as the acidic and basic amino acids. Polar amino acids include, without limitation, asparagine, cysteine, glutamine, histidine, selenocysteine, serine, threonine, tryptophan and tyrosine. Examples of hydrophobic or non-polar amino acids include those residues having nonpolar aliphatic side chains, such as, without limitation, leucine, isoleucine, valine, glycine, alanine, proline, methionine and phenylalanine. Examples of basic amino acid residues include those having a basic side chain, such as an amino or guanidino group. Basic amino acid residues include, without limitation, arginine, homolysine and lysine. Examples of acidic amino acid residues include those having an acidic side chain functional group, such as a carboxy group. Acidic amino acid residues include, without limitation aspartic acid and glutamic acid. Aromatic amino acids include those having an aromatic side chain group. Examples of aromatic amino acids include, without limitation, biphenylalanine, histidine, 2-napthylalananine, pentafluorophenylalanine, phenylalanine, tryptophan and tyrosine. It is noted that some amino acids are classified in more than one group, for example, histidine, tryptophan and tyrosine are classified as both polar and aromatic amino acids. Amino acids may further be classified as non-charged or charged (positively or negatively) amino acids. Examples of positively charged amino acids include without limitation lysine, arginine and histidine. Examples of negatively charged amino acids include without limitation glutamic acid and aspartic acid. Additional amino acids that are classified in each of the above groups are known to those of ordinary skill in the art.

“Equivalent amino acid” means an amino acid which may be substituted for another amino acid in the peptide compounds according to the invention without any appreciable loss of function. Equivalent amino acids will be recognized by those of ordinary skill in the art. Substitution of like amino acids is made on the basis of relative similarity of side chain substituents, for example regarding size, charge, hydrophilicity and hydrophobicity as described herein. The phrase “or an equivalent amino acid thereof” when used following a list of individual amino acids means an equivalent of one or more of the individual amino acids included in the list.

In the embodiments wherein R is a Cn alkyl and Y is —NH₂, the compounds formula (I) having the structure RC(O)—Xaa-Xbb-Y may be named as follows:
Cn C(O)-Xaa-gamma-aminobutyric acid,
or
Cn+1-Xaa-gamma-aminobutyric acid (abbreviated Cn+1-Xaa-GABA).

For example, the compound C12-Asn-GABA described in WO 2018/197666 A1 may be named:
C11 C(O)-Asn-gamma-aminobutyric acid

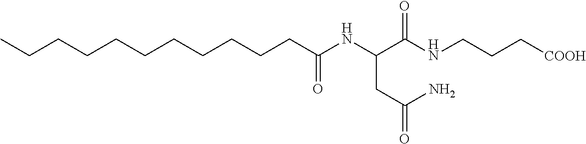 [SMILES: CCCCCCCCCCCC(=O)NC(CC(N)=O)C(=O)NCCCC(=O)O]

In an embodiment, the compound is selected from C16LeuGABA, C16PheGABA, C12AsnGABA, C16GluGABA, C14AsnGABA, C12IleGABA, C14IleGABA and C12AlaGABA.

In another embodiment, the compound is selected from C16PheGABA, C12AsnGABA, C16GluGABA, C14AsnGABA, C12IleGABA, C14IleGABA and C12AlaGABA.

In an embodiment, the compound is C15C(O)-Phe-gamma-aminobutyric acid (or C16-Phe-gamma-aminobutyric acid) In another embodiment, the compound is C11C(O)-Ile-gamma-aminobutyric acid (or C12-Ile-gamma-aminobutyric acid) In another embodiment, the compound is C13C(O)-Ile-gamma-aminobutyric acid (or C14C(O)-Ile-gamma-aminobutyric acid) In another embodiment, the compound is C11 C(O)-Ala-gamma-aminobutyric acid (or C12-Ala-gamma-aminobutyric acid).

In another embodiment, the compound is C15 C(O)-Leu-gamma-aminobutyric acid (or C16-Leu-gamma-aminobutyric acid).

The compound of the invention may be obtained by direct purification from any bacterium containing the pks island—for example from Ligilactobacillus murinus, in particular Ligilactobacillus murinus strain IRSD_2020, or by any one of synthetic chemical method which is well known from the one skilled in the art.

For example, the lipopeptide compounds of interest may be recovered from either the culture medium or the bacterium cell lysates. Typically, the lipopeptide compounds of the invention are isolated from Ligilactobacillus murinus. For example, the lipopeptide compound of the invention may be isolated from Ligilactobacillus murinus strain IRSD_2020. Bacteria employed in production of the lipopeptides of interest can be disrupted by various physical or chemical means, such as freeze-thaw cycling, sonication, mechanical disruption, or cell-lysing agents.

Purification of the lipopeptide of interest from bacteria may be preferred. The procedures described in the Example are exemplary of suitable procedures for purification of lipopeptide compounds from the supernatant of Ligilactobacillus murinus strain IRSD_2020.

The following procedures are exemplary of suitable purification procedures of lipopeptide compound: include: lipopeptide fractionation with spectrometry coupled on-line to a liquid chromatography (as used in the example); reverse phase HPLC; chromatography on silica or on a cation-exchange resin such as DEAE; chromatofocusing; SDS-PAGE; ammonium sulfate precipitation; gel filtration using, for example, Sephadex G-75; Protein A Sepharose columns to remove contaminants; and metal chelating columns to bind epitope-tagged forms of the lipopeptide of interest.

The purification step(s) selected will depend on the nature of the production process used and the particular lipopeptide compound produced.

The protocol for the production of the lipopeptide compound, by means of bacteria culture is also described in “Screening concepts, characterization and structural analysis of microbial-derived bioactive lipopeptides: a review.” Crit Rev Biotechnol. 2017 May; 37(3):393-410. doi: 10.3109/07388551.2016.1163324.

In certain embodiments, the lipopeptide compound of the invention may be synthesised through conventional techniques of chemical synthesis.

### Method for Treating Pain Disorder

In a second aspect, the invention relates to a lipopeptide compound according to the invention for use as a drug, in particular to a lipopeptide compound according to the invention for use in the treatment of a pain disorder, such as a somatic pain or a visceral pain.

Accordingly, an object of the present invention relates to a method of treating pain, in particular a pain disorder such as somatic pain or visceral pain in a subject thereof, the method comprising administering the subject a therapeutically effective amount of a lipopeptide compound of the invention.

Accordingly, an object of the present invention relates to the use of a lipopeptide compound according to the invention in the manufacture of a medicament, in particular of a medicament for the treatment of pain disorder, such as somatic pain or visceral pain.

It will be appreciated that in the second aspect of present invention, unless the context clearly dictates otherwise, referring to “the method of the present invention” is equivalent to referring the second aspect of the invention as a whole.

It will be appreciated that in the second and third aspect of present invention, unless the context clearly dictates otherwise, the expressions “lipopeptide compound according to the invention” refers to a lipopeptide compound as described in the first aspect of the invention or a pharmaceutical acceptable salt thereof. The singular forms “a”, and “the”, referring to the lipopeptide compound include both singular and plural referents.

The lipopeptide compound is preferably selected from C15 C(O)-Phe-gamma-aminobutyric acid, C11 C(O)-Ile-gamma-aminobutyric acid, C13 C(O)-Ile-gamma-aminobutyric acid, C11 C(O)-Ala-gamma-aminobutyric acid, C15 C(O)-Leu-gamma-aminobutyric acid, or a pharmaceutical acceptable salt thereof.

In a preferred embodiment, the lipopeptide compound is C15 C(O)-Phe-gamma-aminobutyric acid, or a pharmaceutical acceptable salt thereof.

Treatment may be for any purpose, including the therapeutic treatment of subjects suffering from pain, as well as the prophylactic treatment of subjects who do not suffer from pain (e.g., subjects identified as being at high-risk pain). As used herein, the terms “treatment,” “treat,” and “treating” refer to reversing, alleviating, inhibiting the progress of a disease or disorder as described herein (i.e., pain), or delaying, eliminating or reducing the incidence or onset of a disorder or disease as described herein, as compared to that which would occur in the absence of the measure taken. The terms “prophylaxis” or “prophylactic use” and “prophylactic treatment” as used herein, refer to any medical or public health procedure whose purpose is to prevent the disease herein disclosed (i.e., pain). As used herein, the terms “prevent”, “prevention” and “preventing” refer to the reduction in the risk of acquiring or developing a given condition (i.e., pain), or the reduction or inhibition of the recurrence or said condition (i.e., pain) in a subject who is not ill, but who has been or may be near a subject with the condition (i.e., pain).

The method of the present invention is suitable in the treatment of a wide range of pain disorders, particularly acute pain, chronic pain, neuropathic pain, inflammatory pain, iatrogenic pain-including cancer pain, infectious pain including herpetic pain visceral pain-, central pain, disorders of pain dysfunction including fibromyalgia, nociceptive pain including post-surgical pain, and mixed pain types involving the viscera, gastrointestinal tract, cranial structures, musculoskeletal system, spine, urogenital system, cardiovascular system and CNS, including cancer pain, back and orofacial pain.

Pain is generally classified as acute or chronic. Acute pain begins suddenly and is short-lived (usually twelve weeks or less). It is usually associated with a specific cause such as a specific injury and is often sharp and severe. It is the kind of pain that can occur after specific injuries resulting from surgery, dental work, a strain or a sprain. Acute pain does not generally result in any persistent psychological response. In contrast, chronic pain is long-term pain, typically persisting for more than three months and leading to significant psychological and emotional problems. Common examples of chronic pain are neuropathic pain (e.g., painful diabetic neuropathy, postherpetic neuralgia), carpal tunnel syndrome, back pain, headache, cancer pain, arthritic pain and chronic post-surgical pain. Clinical pain is present when discomfort and abnormal sensitivity feature among the patient's symptoms. Patients tend to be quite heterogeneous and may display various pain symptoms. Such symptoms include: 1) spontaneous pain which may be dull, burning, or stabbing; 2) exaggerated pain responses to noxious stimuli (hyperalgesia); and 3) pain produced by normally innocuous stimuli. Although patients suffering from various forms of acute and chronic pain may have similar symptoms, the underlying mechanisms may be different and may, therefore, require different treatment strategies. Pain can also be divided into a number of different subtypes according to the pathophysiology, including nociceptive, neuropathic and inflammatory pain.

Nociceptive pain is induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 1994, Textbook of Pain, 13-44). The activation of nociceptors activates two types of afferent nerve fibres.

Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from central nervous system trauma, strains/sprains, burns, myocardial infarction and acute pancreatitis, postoperative pain (pain following any type of surgical procedure), posttraumatic pain, renal colic, cancer pain and back pain. Cancer pain may be chronic pain such as tumour related pain (e.g., bone pain, headache, facial pain or visceral pain) or pain associated with cancer therapy (e.g., postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

There are two types of nociceptive pain: “somatic” pain and “visceral” pain.

“Somatic” pain is caused by injury to skin, muscles, bone, joint, and connective tissues. Deep somatic pain is usually described as dull or aching, and localized in one area. Somatic pain from injury to the skin or the tissues just below it often is sharper and may have a burning or pricking quality. Generally, “Somatic” pain is well-localized pain that results from the activation of peripheral nociceptors without injury to the peripheral nerve or central nervous system.

Somatic pain often involves inflammation of injured tissue. Although inflammation is a normal response of the body to injury, and is essential for healing, inflammation that does not disappear with time can result in a chronically painful disease. The joint pain caused by rheumatoid arthritis may be considered an example of this type of somatic nociceptive pain.

“Visceral” pain refers to pain that originates from ongoing injury to the internal organs or the tissues that support them. When the injured tissue is a hollow structure, like the intestine or the gall bladder, the pain often is poorly localized and cramping. When the injured structure is not a hollow organ, the pain may be pressure-like, deep, and stabbing. Visceral pain could also be associated with a subtype of inflammation (see below)

Neuropathic pain is currently defined as pain initiated or caused by a primary lesion or dysfunction in the nervous system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Woolf and Mannion, 1999, Lancet, 353, 1959-1964). The symptoms of neuropathic pain are difficult to treat, as they are often heterogeneous even between patients with the same disease (Woolf & Decosterd, 1999, Pain Supp., 6, S141-S147; Woolf and Mannion, 1999, Lancet, 353, 1959-1964). They include spontaneous pain, which can be continuous, and paroxysmal or abnormal evoked pain, such as hyperalgesia (increased sensitivity to a noxious stimulus) and allodynia (sensitivity to a normally innocuous stimulus).

The inflammatory process is a complex series of biochemical and cellular events, activated in response to tissue injury or the presence of foreign substances, which results in swelling and pain (Levine and Taiwo, 1994, Textbook of Pain, 45-56). Arthritic pain is the most common inflammatory pain. Rheumatoid disease is one of the commonest chronic inflammatory conditions in developed countries and rheumatoid arthritis is a common cause of disability. The exact aetiology of rheumatoid arthritis is unknown, but current hypotheses suggest that both genetic and microbiological factors may be important (Grennan & Jayson, 1994, Textbook of Pain, 397-407). It has been estimated that almost 16 million Americans have symptomatic osteoarthritis (OA) or degenerative joint disease, most of whom are over 60 years of age, and this is expected to increase to 40 million as the age of the population increases, making this a public health problem of enormous magnitude (Houge & Mersfelder, 2002, Ann Pharmacother., 0.36, 679-686; McCarthy et al., 1994, Textbook of Pain, 387-395). Most patients with osteoarthritis seek medical attention because of the associated pain. Arthritis has a significant impact on psychosocial and physical function and is known to be the leading cause of disability in later life. Ankylosing spondylitis is also a rheumatic disease that causes arthritis of the spine and sacroiliac joints. It varies from intermittent episodes of back pain that occur throughout life to a severe chronic disease that attacks the spine, peripheral joints and other body organs.

Another type of inflammatory pain is visceral pain which includes pain associated with inflammatory bowel disease (IBD). Visceral pain is pain associated with the viscera, which encompasses the organs of the abdominal cavity. These organs include the sex organs, spleen and part of the digestive system. Pain associated with the viscera can be divided into digestive visceral pain and non-digestive visceral pain. Commonly encountered gastrointestinal (Gl) disorders inducing pain include functional bowel disorder (FBD) and inflammatory bowel disease (IBD). These Gl disorders include a wide range of disease states that are currently only moderately controlled including in respect of FBD-, gastro-esophageal reflux, dyspepsia, irritable bowel syndrome (IBS) and functional abdominal pain syndrome (FAPS) and—in respect of IBD—Crohn's disease, ileitis and ulcerative colitis, all of which regularly produce visceral pain. Other types of visceral pain include the pain associated with dysmenorrhea, cystitis and pancreatitis and pelvic pain.

As used herein, the term “cystitis” is a term for a variety of conditions characterized by an inflammation of the bladder. It includes but is not limited to interstitial cystitis, radiation cystitis, recurrent cystitis.

“Interstitial cystitis”, also called bladder pain syndrome, usually refers a condition resulting in recurring discomfort or pain in the bladder or surrounding pelvic region. The major symptoms are pain in the pelvic area and urgent need to urinate often (up to 60 times a day). The causes of IC remain unclear but can include allergy, vascular (blood vessel) disease, autoimmune disease, defects in the lining of the bladder wall, presence of abnormal substances in the urine, unusual types of infections that are not found with standard tests.

“Radiation cystitis” usually refers to a condition resulting in destruction to the normal anatomy of the urinary bladder at the cellular level after the use of radiation in the treatment of multiple cancer types, including, most commonly, pelvic cancers.

“Recurrent cystitis” refers to a condition usually defined as three episodes of urinary tract infection in the previous 12 months, or two episodes in the previous 6 month.

In some embodiments the method of the present invention is particularly suitable for the treatment of visceral pain resulting from cystitis, as detailed below.

In some embodiments the method of the present invention is particularly suitable for the treatment of visceral pain resulting from gastrointestinal disorders, including functional bowel disorder (FBD) and inflammatory bowel disease (IBD) gastro-oesophageal reflux, dyspepsia, irritable bowel syndrome (IBS) and functional abdominal pain syndrome (FAPS), and, in respect of IBD, Crohn's disease, ileitis, ulcerative colitis dysmenorrhea, cystitis and pancreatitis and pelvic pain.

In a specific embodiment, visceral pain is selected from the group consisting of Inflammatory Bowel Diseases (IBD) or Irritable Bowel Syndrome (IBS).

As used herein, the term “Irritable Bowel Syndrome (IBS)” is a term for a variety of pathological conditions causing discomfort in the gastro-intestinal tract. It is a functional bowel disorder characterized by chronic abdominal pain, discomfort, bloating, and alteration of bowel habits in the absence of any organic cause. It also includes some forms of food-related visceral hypersensitivity, such as Gluten hypersensitivity (i.e., Celiac disease).

As used herein, the term “inflammatory bowel diseases (IBD)” is a group of inflammatory diseases of the colon and small intestine. The major types of TBD are Crohn's disease, ulcerative colitis and pouchitis.

In some embodiments the method of the present invention is particularly suitable for the treatment of visceral pain resulting from urogenital disorders.

As used herein, the term “urogenital disorders” refers to conditions affecting the urinary tract and/or the genital tract (i.e., reproductive organs).

As used herein, the term “urologic disorders” refers to conditions affecting the urinary tract, i.e. kidneys, ureters, or bladder. Urologic disorders include but are not limited to bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), urethral diverticulum, chronic urethral syndrome, urethral carbuncle, and urethral stricture. As used herein, the term “genital disorders” refers to conditions affecting the genital tract, Genital disorders include but are not limited to as testicular torsion, prostatitis, peyronie disease.

In some embodiments the method of the present invention is particularly suitable for the treatment of visceral pain resulting from urologic disorders for example selected from the group including bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), urethral diverticulum, chronic urethral syndrome, urethral carbuncle, and urethral stricture.

In some embodiments the method of the present invention is particularly suitable for the treatment of visceral pain resulting from cystitis, preferably from interstitial cystitis.

In some embodiments, the method of the present invention is particularly suitable for the treatment of acute and/or chronic pain which results from cystitis, preferably from interstitial cystitis.

In some embodiments, the method of the present invention is particularly suitable for the treatment of chronic pain which results from cystitis, preferably from interstitial cystitis.

In some embodiments, the method of the present invention is particularly suitable for the treatment of chronic pain which results from cystitis, preferably from interstitial cystitis.

In some embodiments, the method of the present invention is for the treatment of chronic pain which results from cystitis, preferably from interstitial cystitis, and the lipopeptide compound is selected from C15 C(O)-Phe-gamma-aminobutyric acid, C11 C(O)-Ile-gamma-aminobutyric acid, C13 C(O)-Ile-gamma-aminobutyric acid, C11 C(O)-Ala-gamma-aminobutyric acid, C15 C(O)-Leu-gamma-aminobutyric acid or a pharmaceutical acceptable salt thereof.

In some embodiments, the method of the present invention is for the treatment of chronic pain which results from cystitis, preferably from interstitial cystitis, and the lipopeptide compound is C15 C(O)-Phe-gamma-aminobutyric acid or a pharmaceutical acceptable salt thereof.

In some embodiments, the method of the present invention is suitable for the treatment of somatic pain caused by injury to skin, muscles, bone, joint, and connective tissues, such as superficial pain (from the skin or subcutaneous tissue) and deep pain (from deeper structures of the body wall).

In some embodiments, the method of the present invention is particularly suitable for the treatment of chronic pain which results from musculoskeletal disorders such as osteoarthritis/degenerative joint disease/spondylosis, rheumatoid arthritis, Lyme disease, Reiter syndrome, disk herniation/facet osteoarthropathy, fractures/compression fracture of lumbar vertebrae, faulty or poor posture, fibromyalgia, polymyalgia rheumatica, mechanical low back pain, chronic coccygeal pain, muscular strains and sprains, pelvic floor myalgia (levator ani spasm), piriformis syndrome, rectus tendon strain, hernias (e.g. obturator, sciatic, inguinal, femoral, spigelian, perineal, umbilical), abdominal wall myofascial pain (trigger points), chronic overuse syndromes (e.g., tendinitis, bursitis); Neurological disorders such as, brachial plexus traction injury, cervical radiculopathy, thoracic outlet syndrome, spinal stenosis, arachnoiditis syndrome, metabolic deficiency myalgias, polymyositis, neoplasia of spinal cord or sacral nerve, cutaneous nerve entrapment in surgical scar, postherpetic neuralgia (shingles), neuralgia (e.g., iliohypogastric, ilioinguinal, or genitofemoral nerves), polyneuropathies, polyradiculoneuropathies, mononeuritis multiplex, chronic daily headaches, muscle tension headaches, migraine headaches, temporomandibular joint dysfunction, temporalis tendonitis, sinusitis, atypical facial pain, trigeminal neuralgia, glossopharyngeal neuralgia, nervus intermedius neuralgia, sphenopalatine neuralgia, referred dental or temporomandibular joint pain, abdominal epilepsy, abdominal migraine; urologic disorders such as bladder neoplasm, chronic urinary tract infection, interstitial cystitis, radiation cystitis, recurrent cystitis, recurrent urethritis, urolithiasis, uninhibited bladder contractions (detrusor-sphincter dyssynergia), urethral diverticulum, chronic urethral syndrome, urethral carbuncle, prostatitis, urethral stricture, testicular torsion, peyronie disease; gastrointestinal disorders such as chronic visceral pain syndrome, gastroesophageal reflux, peptic ulcer disease, pancreatitis, chronic intermittent bowel obstruction, colitis, chronic constipation, diverticular disease, inflammatory bowel disease, irritable bowel syndrome; reproductive disorders (extrauterine) such as endometriosis, adhesions, adnexal cysts, chronic ectopic pregnancy, chlamydial endometritis or salpingitis, endosalpingiosis, ovarian retention syndrome (residual ovary syndrome), ovarian remnant syndrome, ovarian dystrophy or ovulatory pain, pelvic congestion syndrome, postoperative peritoneal cysts, residual accessory ovary, subacute salpingo-oophoritis, tuberculous salpingitis; reproductive disorders (uterine) such as adenomyosis, chronic endometritis, atypical dysmenorrhea or ovulatory pain, cervical stenosis, endometrial or cervical polyps, leiomyomata, symptomatic pelvic relaxation (genital prolapse), intrauterine contraceptive device; psychological disorders such as bipolar personality disorders, depression, porphyria, sleep disturbances; and other conditions such as cardiovascular disease (e.g., angina), peripheral vascular disease and chemotherapeutic, radiation, or surgical complications.

In some embodiments the method of the present invention is suitable for the treatment of pain which results from autoimmune diseases including multiple sclerosis, neurodegenerative disorders, neurological disorders including epilepsy and senso-motor pathologies, osteoarthritis, rheumatoid arthritis, neuropathological disorders, pain associated with dysmenorrhea, pelvic pain, cystitis, pancreatitis, migraine, cluster and tension headaches, diabetic neuropathy, peripheral neuropathic pain, sciatica, causalgia, and conditions of lower urinary tract dysfunction.

In some embodiments, the prophylactic methods of the invention are particularly suitable for subjects who are identified as at high risk for pain. Typically subject that are risk for pain include patient that will have a surgical operation.

Said compound of the present invention can be used as a drug, in particular as analgesic. The compounds are useful for example in the treatment of pain disorders such as chronic pain and visceral pain.

The skilled man in the art can easily evaluate the analgesic properties of the amino lipid compound of the invention by assessing the capacity of said compound to inhibit neuronal activation of sensory nerves via the GABA_{B} receptor by means of calcium signaling studies in sensory neurons. The analgesic properties may be also tested by neuronal electrophysiology in vitro, axonal recording ex vivo, using animal model of pain (see N Gregory, A L Harris, CR Robinson, P M Dougherty, P N Fuchs, and KA Sluka. An overview of animal models of pain: disease models and outcome measures. J Pain. 2013 November; 14(11)). Typically, the tests that may be used to assess the analgesic activity of a lipopeptide compound are described in the Examples (FIG. 4 and FIG. 6). In a second step, the ability to cross the epithelial barrier can also be tested in vitro in a transwell system, in vivo by a direct quantification of the lipolipide in different organs (see the test described in Example and FIGS. 5 and 6) or ex vivo by the use of using chamber (see H. J. Galipeau & E. F. Verdu. The complex task of measuring intestinal permeability in basic and clinical science. Neurogastroenterol Motil (2016) 28, 957-965).

It will be understood that the daily dose of the compounds and the composition of the present invention will be decided by the attending physician within the scope of sound medical judgment. The specific therapeutically effective dose for any particular patient will depend upon a variety of factors including the type and severity of the disorder to treat; activity of the specific compound employed; the specific composition employed, the age, body weight, general health, sex and diet of the patient; the time and route of administration and the rate of excretion of the specific compound employed; the duration of the treatment; drugs used in combination or coincidental with the specific lipopeptide employed and other factors well known in the medical arts. For example, within the skill of the art it is recommended to start the treatment with doses of the compound at levels lower than those required to achieve the desired therapeutic effect and to gradually increase the dosage until the desired effect is achieved.

The compound of the invention may be administered by any suitable route of administration. For example, the compound according to the invention it can be administered by oral (including buccal and sublingual), rectal, nasal, topical, pulmonary, vaginal, or parenteral (including intramuscular, intra-arterial, intrathecal, subcutaneous and intravenous).

In a preferred embodiment of the invention, the therapeutic composition containing the compound of the invention is administered intrarectally, topically or orally. A rectal administration preferably takes place in the form of a suppository, enema or foam. Intrarectal administration is particularly suitable for intestinal diseases which affect the lower intestinal sections, for example the colon.

### Pharmaceutical Composition

In a third aspect, the invention relates to a pharmaceutical composition, comprising a compound according to the invention and one or more pharmaceutically acceptable excipients

The inhibitors of the present invention, together with one or more conventional adjuvants, carriers, or diluents may be placed into the form of pharmaceutical compositions and unit dosages.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The pharmaceutical composition and unit dosage forms may comprise conventional ingredients in conventional proportions, with or without additional active compounds or principles, and the unit dosage forms may contain any suitable effective amount of the active ingredients commensurate with the intended daily dosage range to be employed. The pharmaceutical composition may be employed as solids, such as tablets or filled capsules, semisolids, powders, sustained release formulations, or liquids such as solutions, suspensions, emulsions, elixirs, or filled capsules for oral use; or in the form of suppositories for rectal administration; or in the form of sterile injectable solutions for parenteral uses. Formulations containing about one (1) milligram of active ingredient or, more broadly, about 0.01 to about one hundred (100) milligrams, per tablet, are accordingly suitable representative unit dosage forms.

The compound of the present invention may be formulated in a wide variety of oral administration dosage forms. The pharmaceutical compositions and dosage forms may comprise compounds of the present invention or pharmaceutically acceptable salts thereof as the active component. The pharmaceutically acceptable carriers may be either solid or liquid. Solid form preparations include powders, tablets, pulls, capsules, cachets, suppositories, and dispersible granules. A solid carrier may be one or more substances which may also act as diluents, flavouring agents, solubilizers, lubricants, suspending agents, binders, preservatives, tablet disintegrating agents, or an encapsulating material. In powders, the carrier generally is a finely divided solid, which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch gelatine, tragacanth, methylcellulose sodium carboxymethylcellulose, a low melting wax, cocoa butter, and the like.

The term “preparation” is intended to include the formulation of the active compound with an encapsulating material as carrier, providing a capsule in which the active component, with or without carriers, is surrounded by a carrier, which is in association with it. Similarly, cachets and lozenges are included. Tablets, powders, capsules, pulls, cachets, and lozenges may be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions may be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavours, stabilizers, and thickening agents. Aqueous suspensions can be prepared by dispersing the finely divided active component in water with viscous material, such as natural or synthetic gums, resins, methylcellulose, sodium carboxymethylcellulose, and other well-known suspending agents. Solid form preparations include solutions, suspensions, and emulsions, and may contain, in addition to the active component, colorants, flavours, stabilizers, buffers, artificial and natural sweeteners, dispersants, thickeners, solubilising agents, and the like.

The compound of the present invention may be formulated for parenteral administration (e.g., by injection, for example bolus injection or continuous infusion) and may be presented in unit dose form in ampoules, pre-filled syringes small volume infusion or in multi-dose containers with an added preservative. The compositions may take such forms as suspensions, solutions, or emulsions in oily or aqueous vehicles, for example solutions in aqueous polyethylene glycol. Examples of oily or non-aqueous carriers, diluents solvents or vehicles include propylene glycol, polyethylene glycol, vegetable oils (e.g., olive oil, and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilization from solution for constitution before use with a suitable vehicle, e.g., sterile, pyrogen-free water.

The pharmaceutical composition of the second aspect of present invention may of course be used in a method or use according to the second aspect of the invention.

### Synthesis of the Lipopeptide of the Invention

In a fourth aspect, the present invention also relates to a method to obtain a compound of the invention, characterized in that the compound is synthesized using peptide solid phase synthesis techniques or liquid phase synthesis technique.

The “peptide solid phase synthesis techniques” means synthesis strategies which are well known by the skilled person in the art, and can be found in peptide synthesis dedicated books such as: Paul Lloyd-Williams, Fernando Albericio, Ernest Giralt, “Chemical Approaches to the Synthesis of Peptides and Proteins”, CRC Press, 1997 or Houben-Weyl “Methods of Organic Chemistry, Synthesis of Peptides and Peptidomimetics”, Vol E 22a, Vol E 22b, Vol E 22c, Vol E 22d., M. Goodmann Ed., Georg Thieme Verlag, 2002 or in the review article Ann M. Thayer “Making Peptides At Large Scale”. Chemical & Engineering News May 30, 2011 Volume 89, Number 22 pp. 21-25.

The “liquid phase synthesis technique” is classical organic synthesis methods which are well known from the one skilled in the art.

For the production of peptide at large scale (between 10 grams and 2 tons), it could be preferred to use liquid phase synthesis technique.

Accordingly, in a specific embodiment the method to obtain a compound of the invention is characterized in that the compound of formula (I) is synthesized using liquid phase synthesis technique.

For example, the synthesis of compound according to the invention may be performed as described in WO 2018/197666 A1, which describes that the compound C12-Asn-GABA (not according to the present invention) may be performed starting from GABA (γ-Aminobutyric Acid) following 7 different steps which are summarized in the scheme below:

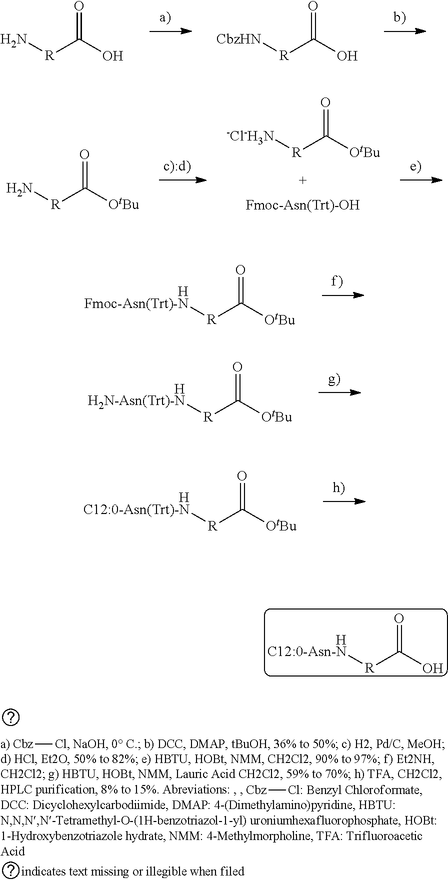

The details of preparation are described below.

1) CBz-GABA-OH: A solution of γ-aminobutyric acid (5.22 g, 50.6 mmol, 1 eq) in 2M NaOH solution (25 mL, 50 mmol, 1 eq) was cooled to 0° C. and treated with benzyl chloroformate (8.23 mL, 55.6 mmol, 1.15 eq), while pH is maintained around 10 by continuous addition of 3M NaOH solution. After 15 minutes, the reaction was allowed to stir at room temperature for 3 hours. After two extractions with Et₂O, the pH of the aqueous solution was adjusted to 1.5 by addition of 6M HCl solution. After having saturated with solid NaCl, the aqueous layer was extracted with EtOAc (3×50 mL). The combined organic layers were washed with brine (3×25 mL), dried over MgSO₄ and evaporated under vacuum. The oily residue was taken up with Et₂O and the solvent removed again to give a white solid of CBz-GABA-OH (11.3 g) used directly in the next step.

2) CBz-GABA-OtBu: To a solution of CBz-GABA-OH (11.3 g), tBuOH (14.5 mL, 152 mmol, 3 eq) and DMAP (620 mg, 5.1 mmol, 0.1 eq) in CH₂Cl₂, after cooling at 0° C., was added dicyclohexylcarbodiimide (12.52 g, 60.7 mmol, 1.2 eq). After one hour, the reaction was stirred vigorously overnight at room temperature. DCU was filtered and washed with EtOAc (2×5 mL). The filtrate was washed with 1M HCl solution (50 mL), and refiltrated. The aqueous layer was extracted with CH₂Cl₂ (3×50 mL). The combined organic layers were washed with brine (50 mL), 5% NaHCO₃ solution (50 mL) and brine (50 mL) again. The layer was dried over MgSO₄ and evaporated under vacuum. The crude was purified by column chromatography (pentane/AcOEt 90/10 to 70/30) to obtain CBz-GABA-OtBu (7.39 g, 50%). MS (ESI+) [M+Na]⁺: 316.17; [2M+Na]⁺: 609.00; MS (ESI−) [M]⁻: 293.33.

3) GABA-OtBu·HCl: A solution of CBz-GABA-OtBu (2 g, 6.82 mmol, 1 eq) in MeOH (20 mL) was treated with Pd/C (10%, 200 mg, 10% w/w). After 6 hours, the catalyst was filtered over a Celite® pad. The solvent was carefully evaporated (caution, final product is volatile). 1M HCl solution (50 mL) was added. The aqueous layer was extracted with EtOAc (2×50 mL) and pH was adjusted to 10 by addition of NaOH pellets. The aqueous layer was extracted with EtOAc (3×50 mL). This last organic phase was washed with brine (2×25 mL), dried over MgSO₄ and carefully evaporated. The crude reaction mixture was dissolved in Et₂O (25 mL) and pH was adjusted to 1 by addition of 2M HCl solution in Et₂O. After 30 minutes of stirring, 676 mg GABA-OtBu·HCl (51% yield) was filtered and dried.

4) Fmoc-Asn(Trt)-GABA-OtBu: To a solution of Fmoc-Asn(Trt)-OH (328 mg, 0.55 mmol, 1.1 eq.) and GABA-OtBu·HCl (98 mg, 0.5 mmol, 1 eq.) in CH₂Cl₂ (4 mL) was added HBTU (176 mg, 0.75 mmol, 1.5 eq), HOBt (7 mg, 0.05 mmol, 0.1 eq) and N-methyl morpholine (176 μL, 1.6 mmol, 3.2 eq). The mixture was stirred overnight. The solvent was evaporated in presence of silica gel. The product was purified by column chromatography (CH₂Cl₂/MeOH 97.5/2.5) to obtain Fmoc-Asn(Trt)-GABA-OtBu (360 mg, 97%). MS (ESI+) [M+H]⁺: 738.17; [M+Na]⁺: 760.50; MS (ESI−) [M-Fmoc]⁻: 514.58.

5) Asn(Trt)-GABA-OtBu: To a solution of Fmoc-Asn(Trt)-GABA-OtBu (350 mg, 0.47 mmol, 1 eq) in CH₂Cl₂ (1.35 mL) was added diethylamine (1.35 mL) and stirred for 2 h at room temperature. The resulting solution was concentrated in vacuum and used directly in the amine coupling step.

6) C12:0-Asn(Trt)-GABA-OtBu: To a solution of Asn(Trt)-GABA-OtBu (0.47 mmol, 1 eq.) and lauric acid (143 mg, 0.71 mmol, 1.5 eq.) in CH₂Cl₂ (4 mL) was added HBTU (194 mg, 0.83 mmol, 1.75 eq), HOBt (6 mg, 0.04 mmol, 0.1 eq) and N-methyl morpholine (195 μL, 1.77 mmol, 3.75 eq). The mixture was stirred overnight. The solvent was evaporated in presence of silica gel. The product was purified by column chromatography (pentane/AcOEt 75/25 to 60/40) to obtain C12:0-Asn(Trt)-GABA-OtBu (235 mg, 71%). MS (ESI+) [M+H]⁺: 698.25; [M+Na]⁺: 720.50; MS (ESI−) [M]⁻: 696.75.

7) C12:0-Asn-GABA-OH: To a solution of C12:0-Asn(Trt)-GABA-OtBu (235 mg, 0.33 mmol, 1 eq) in CH₂Cl₂ (0.5 mL) was added TFA (0.5 mL) and stirred for 3 h at room temperature. The resulting solution was concentrated in a vacuum. Traces of TFA were eliminated by coevaporation with acetonitrile (5×2 mL). The crude reaction mixture was stirred with diisopropyl ether (10 mL) and filtrated. The resulting white solid (102 mg) was purified by HPLC to give 11 mg (8.6%) of C12:0-Asn-GABA-OH. Analytical LC-MS Rt: 1.62 mn; RMN (500 MHz, MeOD): δ=4.64 (t, J=6.9 Hz, 1H); 3.20 (t, J=6.6 Hz, 2H); 2.67 (dd, J=6.0, 15.3 Hz, 1H); 2.58 (dd, J=7.3, 15.3 Hz, 1H); 2.30 (t, J=7.4 Hz, 2H); 2.22 (t, J=7.4 Hz, 2H); 1.76 (quint, J=7.0 Hz, 2H); 1.57 (sl, 2H); 1.27 (sl, 16H), 0.93-0.86 (t, J=6.4 Hz, 3H); HRMS [M+H]⁺ calc: 400.2811 found: 400.2804.

All reactions requiring anhydrous conditions were conducted in flame dried glassware with magnetic stirring under an atmosphere of nitrogen unless otherwise mentioned. Anhydrous CH₂Cl₂ was obtained from the Innovative Technology PS-Micro solvent purification system. Other solvents and reagents were used as obtained from the suppliers (Aldrich, Alfa Aesar, Acros) unless otherwise noted. Reactions were monitored by TLC using plates precoated with silica gel 60 (Merck). Reaction components were visualized by using a 254 nm UV lamp and treatment with basic KMnO₄ solution. Column chromatography was performed by using silica gel 40-63 μm. ES-MS and High-resolution mass data were obtained using the mass spectrometers Synapt G2-S (Waters) operated by the Laboratoire de Mesures Physiques of University Montpellier and were obtained by positive electrospray ionization methods. ¹H NMR spectra were obtained at 300 or 500 MHz on Bruker spectrometers. The spectra were recorded in MeOD. The ¹H NMR spectra are reported as follow: chemical shift in ppm [multiplicity, coupling constant(s) J in Hz, relative integral]. The multiplicities are defined as follow: br.=broad, m=multiplet, s=singlet, d=doublet, t=triplet, q=quadruplet, quint.=quintuplet or combinations thereof. ¹³C NMR spectra were recorded in MeOD. LCMS analysis were carried out on a Waters Micromass with Alliance 2695 chain with a Chromolite HR C18 column (25×4.6 mm, Merck Inc) monitoring at 214 nm with positive mode for mass detection. Solvents for LCMS were water with 0.1% formic acid (solvent A) and acetonitrile with 0.1% formic acid (solvent B). Compounds were eluted at a flow rate of 3 mL/min by a linear gradient of 0% to 100% solvent B over 2.5 min, and finally 100% solvent B for 1 min before equilibrating the column back to 0% solvent B over 1 min.

LC-MS Purification: samples were prepared in DMSO. The LC/MS autopurification system consisted of a binary pump Waters 2525, an injector/fraction collector Waters 2676, coupled to a Waters Micromass ZQ spectrometer (electrospray ionization mode, ESI+). Purifications were carried out using a Luna® 5 μm C18 100 Å, LC Column 100×21.2 mm, AXIA™ Packed. A flow rate of 20 mL/min and a gradient of 40-60% B over 10 min were used. Eluent A: water with 0.1% TFA; eluent B: acetonitrile with 0.1% TFA. Positive ion electrospray mass spectra were acquired at a solvent flow rate of 204 μL/min. Nitrogen was used for both the nebulizing and drying gas. The data were obtained in a scan mode ranging from 100 to 1000 m/z in 0.1 s intervals; 10 scans were summed up to get the final spectrum. Collection control trigger is set on single protonated ion with a MIT (minimum intensity threshold) of 7.105.

The invention is further illustrated by the following figures and examples. However, these examples and figures should not be interpreted in any way as limiting the scope of the present invention.

### FIGURES

FIG. 1. Prenatal stress induces visceral hypersensitivity in the adult offspring. Visceral motor response to colorectal distensions (VMR) in control (white) or prenatal stress (PS; black) mice in response to increasing pressures of distension (15, 30, 45 and 60 mmHg) was measured, in both male (FIG. 1A) and female (FIG. 1B) offspring. Data are expressed as mean±SEM (n=14-19 mice/group, 3 independent experiments). Statistical analysis was performed using two-way Anova analysis of variance and subsequent Sidak's multiple comparison test. *P<0.05, **p<0.01, ***p<0.001 significantly different from control group. The results are also expressed as area under the curve (AUC) presented as scatter dot plot with the mean. Statistical analysis was perform using a Mann-Whitney test. **p<0.01, significantly different from the control group. (FIG. 1C) Heat map of polyunsaturated fatty acid (PUFA) metabolites quantified by LC-MS/MS. Data are shown in a matrix format: each row represents a single PUFA metabolite, and each column represents a group of mice: C M=control males, PS M=PS males, C F=control females, PS F=PS females. Each color patch represents the normalized quantity of PUFA metabolites (row) in a group of mice (column), with a continuum of quantity from bright green (lowest) to bright red (highest). The pattern and length of the branches in the dendrogram on the left, reflect the relatedness of the PUFA metabolites, on top the relatedness of the mouse groups. The dashed red line is the dendrogram distance used to cluster PUFA metabolites and mouse groups. (n=15 mice/group, 2 independent experiments). (FIG. 1D) Heatmap of mRNA expression of genes coding for enzymes implicated in PUFA metabolism (top panel), colonic immune response (middle panel) and gut homeostasis (bottom panel). Data are shown in a matrix format: each row represents a single PUFA metabolite, and each column represents a group of mice: C M=control males, PS M=PS males, C F=control females, PS F=PS females. Each color patch represents the normalized gene expression (row) in a group of mice (column), with a continuum of quantity from bright green (lowest) to bright red (highest). The pattern and length of the branches in the dendrogram on the left, reflect the relatedness of the gene expression on top the relatedness of the mouse groups. The dashed red line is the dendrogram distance used to cluster genes and mouse groups. (n=15 mice/group, 3 independent experiments)

FIG. 2. Prenatal stress induces a gut microbiota dysbiosis both in male and female mice. Linear Discriminant Analysis (LDA) score in male (FIG. 2A) and female (FIG. 2B) PS mice vs. control mice; diversity indices in male (FIG. 2C) and female (FIG. 2D) PS mice vs. control mice; PICRUSt-based predicted gut microbial functions in male (FIG. 2E) and female (FIG. 2F) PS mice vs. control mice. (n=15 mice/group)

FIG. 3. Prenatal stress alters gut microbiota spatial organization in adulthood. (FIG. 3A) Bacteria were labelled with the universal probe Eub338 (red), wheat germ agglutinin-FITC was used to stain the polysaccharide-rich mucus layer (green), and the epithelial cell nucleus was stained with DAPI (blue). (FIG. 3B) Bacteria penetration into the mucus was measured in both male (M, square) and female (F, circle) control (white symbols) and PS (black symbols) mice by image processing on Fiji by quantifying the number of 16S RNA labelled pixel between the edge of the lumen to middle of the mucus (Apical) and between the middle of the mucus to the edge of the epithelium (Basal). The results are expressed as scatter dot plot with the mean. Statistical analysis was perform using a Mann-Whitney test. **p<0.01, significantly different from the control group. (n=12 mice/group; 4 images/mice, 2 independent experiments)

FIG. 4. The abundance of L. animalis is inversely correlated to visceral hypersensitivity in adulthood Multivariate analysis of the complete cohort: (FIG. 4A) Two-dimensional PCA (R2=52%) score plot of data generated from 56 samples (control, n=28, blue; PS, n=28, red). Each dot corresponds to an individual. (FIG. 4B) Two-dimensional PLS-DA (R2X=26.2%, R2Y=77.9%, Q2=0.367) score plot of data generated from 53 samples (control, n=26, blue; PS, n=27, red). Each dot corresponds to an individual. The black ellipse corresponds to a 95% confident interval based on the Hotelling's T². (FIG. 4C) Permutation test for PLS-DA model validation (FIG. 4D) Boxplots of discriminant (Variable Importance in Projection>1) and significant (FDR-corrected p value of Wilcoxon test<0.05) variables. (FIG. 4E) Spearman correlations were used to analyse the correlation between the faecal microbiota abundances and visceral motor response to colorectal distension expressed in AUC, in male (square) and female (circle) offspring. p and r values are indicated on each graph.

FIG. 5. L. murinus IRSD_2020 concentration is decreased in PS mouse feces The levels of the L. murinus strain IRSD_2020 (FIG. 5A) and of the reference L. murinus animalis strain DSMZ 20602 (FIG. 5B) were quantified by TaqMan® real-time PCR in the feces of control mice (white) or PS mice (black), in the male (square) and female (circle) offspring. Data are expressed as scatter dot plot with the mean. Statistical analysis was performed using a Mann-Whitney test. ****p<0.0001, significantly different from the control group (n=27-36 mice/group, 3 independent experiments). Spearman correlations were used to analyze the correlation between the fecal bacteria quantity and visceral motor response to colorectal distension expressed in AUC, in male (square) and female (circle) offspring. P and r values are indicated on each graph.

FIG. 6. L. murinus IRSD_2020 produces an analgesic lipopeptide (FIG. 6A) Concentration of lipopeptides quantified by LC-MS/MS in the bacterial pellets of L. murinus TRSD_2020 cultivated without (white circle) or with GABA (4 mg/mL) (black circle). Data are expressed as scatter dot plot with the mean (n=9). Statistical analysis was performed using a Mann-Whitney test. *p<0.05, **p<0.01, significantly different from the corresponding L. murinus TRSD_2020 without GABA. (FIG. 6B) Concentration of lipopeptides quantified by LC-MS/MS in the colon of male (square) and female (circle) control (white) and PS mice (black). Data are expressed as scatter dot plot with the mean (n=9-10). Statistical analysis was performed using a Mann-Whitney test. **p<0.01, significantly different from the corresponding control group (C) Visceral motor response to colorectal distensions (VMR) in response to increasing pressures of distension (15, 30, 45 and 60 mmHg) was measured, in both male and female PS offspring. Measurements were done before (white) and after intracolonic administrations of C14AsnGABA (black). Data are expressed as mean±SEM (n=19 mice/group, 2 independent experiments). Statistical analysis was performed using two-way Anova analysis of variance and subsequent Sidak's multiple comparison test. *P<0.05, ***p<0.001, ****p<0.0001 significantly different from pretreatment group. The results are also expressed as area under the curve (AUC) presented as scatter dot plot with the mean for male (square) and female (circle) mice. Statistical analysis was perform using a Wilcoxon test. ****p<0.0001, significantly different from the pretreatment group.

FIG. 7. Lipopeptides-GABA concentrations are decreased in the feces of IBS patients. (FIG. 7A) C16LeuGABA (left panel) and C12AsnGABA (right panel) concentrations quantified by LC-MS/MS in the feces of healthy volunteers (n=18; white) and IBS patients (black) with an IBS-SSS corresponding to mild (n=7), moderate (n=18) or severe (n=18) IBS. Data are expressed as scatter dot plots with the mean. Statistical analysis was performed using a Kruskal-Wallis analysis of variance and subsequent Dunn's multiple comparison test. **p<0.01, **p<0.001 significantly different from healthy volunteers. (FIG. 7B) Spearman correlations were used to analyse the correlation between the concentration of CL16LeuGABA and abdominal pain score (left panel) or IBS-SSS (right panel) in healthy volunteers (HV, white) and patients with IBS (black). p and r values are indicated on each graph. (FIG. 7C) Percentage of responding neurons pretreated with increasing amounts of C16LeuGABA or vehicle (HBSS/MeOH 0.06%; 0 μM) and treated with capsaicin (500 nM) or (FIG. 7D) a mix of GPCR agonists (histamine, serotonin, and bradykinin 10 μM each). Data are represented as mean±SEM; n=4 independent experiments of 2-3 wells per condition and 20-50 neurons per well. Statistical analysis was performed using Kruskal-Wallis analysis of variance and subsequent Dunn's post hoc test. *p<0.05, **p<0.01, ****p<0.0001 significantly different from capsaicin or GPCR mix.

FIG. 8. L. murinus IRSD_2020 produces GABA-lipopeptides. Concentration of lipopeptides quantified by LC-MS/MS in the bacterial pellets of L. murinus IRSD_2020 cultivated without (0 mg/mL) or with GABA (2, 4 or 8 mg/mL). Data are expressed as scatter dot plot with the mean (n=9).

FIG. 9. Correlation between C16LeuGABA in feces and SF-36 score. Spearman correlation was used to analyse the correlation between the concentration of CL16LeuGABA and SF-36 score in healthy volunteers (HV, white) and patients with IBS (black). p and r values are indicated on the graph.

FIG. 10. Percentage of responding neurons pretreated with increasing amounts of C12AsnGABA, C14AsnGABA or C16LeuGABA or vehicle (HBSS/MeOH 0.06%; 0 μM) and treated with capsaicin (500 nM). Data are represented as mean±SEM; n=4-5 independent experiments of 3 wells per condition and 20-50 neurons per well. Statistical analysis was performed using Kruskal-Wallis analysis of variance and subsequent Dunn's post hoc test. *p<0.05, ****p<0.0001 significantly different from capsaicin.

### EXAMPLES

### Example 1

L. murinus produces lipopeptide containing GABA with analgesic properties (C12AsnGABA described in Perez-Berezo et al, 2017). L. murinus is probably involved in the maintenance of normal gut sensitivity through the production of its analgesic compounds. L. murinus produces others lipopeptides containing GABA such as C16PheGABA, C12AsnGABA, C16GluGABA, C14AsnGABA, C12IleGABA, C14IleGABA and C12AlaGABA.

Given we previously showed that C12AsnGABA, a lipopeptide containing GABA (the main inhibitory transmitter of the central nervous system), exhibits analgesic properties in visceral pain (WO 2018/197666 A1), the inverse correlation between L. murinus abundance and visceral sensitivity to colorectal distension, led us to hypothesize that this bacterium was implicated in the maintenance of the normosensitivity by producing analgesic molecules. As expected L. murinus, in the presence of GABA, produced GABA-containing lipopeptides, such as C16PheGABA, C12AsnGABA, C16GluGABA, C14AsnGABA, C12IleGABA, C14IleGABA and C12AlaGABA highlighting the functional redundancy of bacteria to produce lipopeptides linked to GABA.

### Example 2

### Summary

Objectives: Clinical studies revealed that early life adverse events contribute to the development of irritable bowel syndrome (IBS) in adulthood. The aim of our study was to investigate the relationship between prenatal stress (PS), gut microbiota and visceral hypersensitivity with a focus on bacterial lipopeptides containing GABA.

Design: We developed a model of PS in mice and evaluated, in adult offspring, visceral hypersensitivity to colorectal distension, colon inflammation, barrier function and gut microbiota taxonomy. We quantified the production of lipopeptides containing GABA by mass spectrometry in a specific strain of bacteria decreased in PS, in PS mouse colons and in feces of IBS patients and healthy volunteers. Finally, we assessed their effect on PS-induced visceral hypersensitivity.

Results: Prenatally stressed mice of both sexes presented visceral hypersensitivity, no overt colon inflammation or barrier dysfunction but a gut microbiota dysbiosis. The dysbiosis was distinguished by a decreased abundance of Ligilactobacillus murinus, in both sexes, inversely correlated with visceral hypersensitivity to colorectal distension in mice. An isolate from this bacterial species produced several lipopeptides containing GABA including C14AsnGABA. Interestingly, intra-colonic treatment with C14AsnGABA decreased the visceral sensitivity of PS mice to colorectal distension. The concentration of C16LeuGABA, a lipopeptide which inhibited sensory neurons activation, was decreased in feces of IBS patients compared to healthy volunteers.

Conclusion: Prenatal stress impacts the gut microbiota composition and metabolic function in adulthood. The reduced capacity of the gut microbiota to produce GABA lipopeptides could be one of the mechanisms linking prenatal stress and visceral hypersensitivity in adulthood.

### Introduction

Irritable bowel syndrome (IBS) affects ˜11% of the world population and is one of the most common causes of gastroenterology consultation [1]. This functional intestinal disorder is characterized by repeated periods of abdominal pain and transit changes (constipation, diarrhea, or an alternate of both). Risk factors for IBS encompass infection, female sex and stress. Indeed, stressful events deeply impact body functions and have been linked to increased IBS symptoms severity [2-4]. Recent literature has shown that early-life adverse events have consequences on the onset of chronic non-communicable diseases in adulthood such as IBS [5-7]. In 2012, Bradford et al. showed that IBS patients reported more early-life stressors than healthy subjects, linking early-life adverse events and IBS onset in adulthood [8]. Because events occurring before birth can hardly be included in questionnaires, the effects of in utero events on intestinal dysfunction and IBS symptoms remain unexplored.

In murine models, stress in pregnancy results in gut microbiota dysbiosis in mother and offspring [9]. In patients with IBS, dysbiosis has been reported but there is a discrepancy in the bacterial composition between studies. For instance, in IBS-D, Tap and colleagues quantified an increase in Bacteroides [10] while Su and collaborators described a decrease in the same IBS subtype [11]. In IBS, a lot of studies investigated the taxonomic composition of the microbiota but few studies have investigated active genes, proteins, or metabolites [12]. However, to better understand the role played by microbiota in host function, studying the molecules produced by the microbiota rather than composition may be more relevant. Indeed, although the taxonomic composition of the human microbiota varies tremendously across individuals, its functional capacity is highly conserved [13]. In a previous study, we highlighted that a probiotic, Escherichia coli Nissle 1917, produces a lipopeptide, the C12-asparagine-□-aminobutyric acid (C12AsnGABA) that decreases both neuronal activation induced by pro-nociceptive molecules in vitro and capsaicin-induced hypersensitivity in vivo [14]. We then determined lipoamino acid (LpAA) and GABA-lipopeptide structures by high resolution mass spectrometry and developed a quantitative method of newly identified LpAA and lipopeptides-GABA by LC-MS/MS in different strains of bacteria [15, 16].

We hypothesized that visceral hypersensitivity in adulthood may originate from functional intestinal microbiota dysbiosis induced by stress in pregnancy. Based on our previous studies demonstrating the ability of lipids to regulate sensory neurons activation [14,17,18], we assumed that bacteria-derived GABA-lipopeptides may be the link between functional dysbiosis and IBS symptoms. We show that prenatal stress induces in adult mouse offspring microbiota dysbiosis characterized by a decrease in GABA-containing lipopeptides and visceral hypersensitivity. This decrease is also observed in feces of patients with IBS.

### Methods

### Animal Experiments

Six to ten-week-old C57BL/6J mice (Janvier, Saint Quentin Fallavier, France) were used. All procedures were performed in accordance with the Guide for the Care and Use of Laboratory Animals of the European Council and were approved by the Animal Care and Ethics Committee of US006/CREFE (CEEA-122; application number APAFIS #16385-CE2018080222083660V3). Mice were raised in sanitary conditions without pathogens, with free access to water and food, and submitted to alternating cycles of 12 hours of light and darkness. After mating (3 males and 2 females per cage), C57BL/6J dams, 2 mice per cage, were randomly assigned to receive stress from day 13 to day 18 of gestation. The pregnant mice assigned to the stress group experienced bright light (100 watts) coupled to restraint in a drilled falcon tubes (50 mL, Fischer Scientific, Illkirch, France) for 30 minutes, 3 times a day, with at least 3 hours between each stress session. Stress efficacy was assessed by controlling fecal output (>4 fecal pellets during the first stress session for the first two days). The pregnant mice assigned to the control group were not manipulated. On the last day, gestating mice were put one per cage for natural delivery. The pups were weighed every three days to monitor their growth. On postnatal day 21 to 28, the pups were weaned from their mothers. The offspring between 8 and 11 weeks of age, both male and female, were assessed for visceral sensitivity to colorectal distension, paracellular permeability, colon inflammation, plasma corticosterone concentration, colonic concentration of GABA-containing lipopeptides and taxonomic, predicted functional analysis and biogeography of the gut microbiota (Supplementary methods). In a second set of experiments, visceral sensitivity to colorectal distension was assessed in PS mice before and 30 min after intracolonic administration of C14AsnGABA (10 μM).

### Patients

IBS-D patients were recruited according to Rome IV criteria [19]. Clinical examination and standard biological tests were normal. Total colonoscopy and additional tests when necessary had excluded organic disease. Demographic data were prospectively recorded (Table 1). IBS symptom severity was assessed by IBS severity scoring system (IBS-SSS) [20]. This score allows to classify patients with mild (75-174), moderate (175-299), or severe symptoms (>300) (Table 1). Healthy volunteers were recruited by public advertisement using the GSRS questionnaire according to European consensus (European cost project) [21]. The research was performed according to the Declaration of Helsinki. All patients gave their written informed consent. Patients were included at the gastroenterology department of the tertiary care center (Rouen University Hospital, France) between March 2017 and June 2019 and at the outpatient clinic of the University Hospitals Leuven in Belgium between January 2019 and October 2021. For French patients, the use of informatics data was declared to the CNIL (no. 817.917) and the biological collection was declared to the French Ministry (no DC 2016-2637 and no AC 2019-3840). Collection of samples from healthy volunteers and IBS patients in Leuven was approved by the Medical Ethics Committee of the University Hospitals Leuven (B) (S51573).

TABLE 1
Demographic and clinical characteristics of the human cohort

| | HV | IBS |
|---|---|---|
| N | 18 | 43 |
| Female, N (%) | 15 (75) | 32 (78) |
| Age, mean ± SD | 31.9 ± 12.1 | 29.6 ± 8.6 |
| Mild IBS, N (%) | | 7 (16) |
| 75 < IBS-SSS < 149 | | |
| IBS-moderate, N (%) | | 18 (42) |
| 150 < IBS-SSS < 299 | | |
| IBS-severe, N (%) | | 18 (42) |
| BBS-SSS > 300 | | |

HV: healthy volunteers,
IBS: irritable bowel syndrome,
SD: standard deviation

### Supplementary Methods

### Colorectal Distension

Nickel-chrome electrodes were implanted in the abdominal external oblique musculature of anesthetized mice in order to detect EMG activity as previously described [Boue et al., «Endogenous regulation of visceral pain via production of opioids by colitogenic CD4(+) T cells in mice», Gastroenterology, vol. 146, no 1, p. 166-175, janv. 2014, doi: 10.1053/j.gastro.2013.09.020.]. CRD was performed 3 days post-surgery by inserting a distension catheter (Fogarty catheter for arterial embolectomy, 4 F; Edwards Lifesciences, Nijmegen, Netherlands) into the colon at 5 mm from the anus. The balloon was progressively inflated in a stepwise of 15 mmHg (from 0 to 60 mmHg) performing 10 s distension for each pressure and with resting intervals of 5 min (n=14-19 mice/group, 3 independent experiments). In a second set of experiments, mice submitted to stress (n=15 per group, 2 independent experiments) were treated with a 100 μL intracolonic injection of C14snGABA (10 μM) or its vehicle (EtOH 40%) 30 min before CRD. The results were expressed as the area under the curve from 15 to 60 mmHg.

### Permeability and Macro and Microscopic Scores

To determine paracellular permeability, mice were gavaged with dextran 4 kDa-FITC (25 mM; Sigma, St. Quentin Fallavier, France). Four hours after, mice were sacrificed. Blood was collected for FITC quantification in the serum and colons were removed. Length and thickness were measured and macroscopic colonic tissue damages were scored from 0 to 3 for the intensity of adhesion and strictures, from 0 to 2 for the intensity of edema, erythema, ulceration and diarrhea and from 0 to 1 (absent or present) for the mucus, hemorrhage and fecal blood; the maximal score being 17. For histological examination, a piece of colon located at 2 cm proximal to the anus was resected, fixed in 10% phosphate buffered formalin, embedded in paraffin, sectioned, and stained with hematoxylin-eosin. Slides were examined and graded for cellular infiltration, mucosal architecture alteration and submucosal oedema from 0 to 3 (absent, mild, moderate and severe) and vasculitis, muscular thickening, crypt abscess and goblet cell depletion from 0 to 1 (absent or present); the maximal score being 13.

### Real Time PCR Analysis

Colon biopsies were crushed in 500 μL of Trizol (Invitrogen, Thermofisher scientific, Saclay, France) in Precellys lysing kit tubes (Bertin Technologies, Montigny le Bretonneux, France) placed in a Precellys (2823 g, 30 seconds twice; Bertin Technologies). After addition of chloroform and centrifugation (15 minutes, 11292 g at 4° C.), the supernatant containing the RNA was removed. Ethanol 70% (vol/vol) was added and the contents were placed in columns (GenElute® mammalian total RNA miniprep kit, Sigma Aldrich). The RNA was extracted according to the manufacturer recommendations. Subsequently the RNA was dosed in a nanodrop (implenGmbH, Dominique Dutscher, Issy-les-Moulineaux, France). RNA (3 g) preparations from colon of mice were used for the total RNA reverse-transcription with Moloney murine leukemia virus reverse transcriptase (Fisher Scientific) using random hexamers (Fisher Scientific) for priming. Transcripts encoding Hypoxanthine phosphoribosyl transferase (Hprt), Trefoil factor 3 (Tff3), Mucin 2 (Muc2), Occludin (Ocln), Zonula occludens-1 (Tjp1), Regenerating islet-derived 3 gamma (Reg3γ), Matrilysin (Mmp7), Tumor necrosis factor alpha (Tnfa), Chemokine (C-Cmotif) ligand 5 (Ccl5), Transforming growth factor beta (Tgfβ), Interleukin 6 (Il6), Interleukine 1 beta (Il1β), Nuclear factor kappa B (Nfκb), Chemokine (C-X-C motif) 2 ligand Cxcl2, Lipoxygenase 15 (Alox15), Lipoxygenase 5 (Alox5), Lipoxygenase 12 (Alox12), Cyclooxygenase 1 (Ptgs1), Cyclooxygenase 2 (Ptgs2), Pro-enkephaline (Penk), Interferon gamma (Ifnγ), Peroxysome proliferator-Activated Receptor (PPARα), Acyl-CoA Thioesterase 12 (Acot12), Angiopoietine like 4 (Angptl4), Cluster of differenciation 36 (CD36), Aryl hydrocarbon receptor (Ahr), glucocorticoid receptor (Nr3C1) and soluble epoxy hydrolase (Ephx2), were quantified by real-time PCR using specific forward and reverse primers, the kit Takyon SYBR 2× MasterMix blue dTTP (Eurogentec) and the LightCycler480II (Roche Diagnostics, Meylan, France). Hierarchical clustering was performed, and heat maps were obtained with R (www.rproject.org). Gene expressions were transformed to z scores and clustered based on 1-Pearson correlation coefficient as distance and the Ward algorithm as agglomeration criterion.

### Liquid Chromatography/Tandem Mass Spectrometry (LC-MS/MS) Measurements

PUFA metabolites were quantified from mice colons by mass spectrometry after lipid extraction as previously described [P. Le Faouder et al., «LC-MS/MS method for rapid and concomitant quantification of pro-inflammatory and pro-resolving polyunsaturated fatty acid metabolites», J. Chromatogr. B Analyt. Technol. Biomed. Life. Sci., vol. 932, p. 123-133, août 2013, doi: 10.1016/j.jchromb.2013.06.014.]. After the addition of 500 μL of PBS, and 5 μL deuterated Internal Standard (IS) mixture (5-HETEd8, LxA4d4 and LtB4d4), the colons were crushed in lysing MatrixA tubes in a precellys (Bertin Technologies). After two crush cycles (6.5 ms-1, 30 s), 10 μL of suspensions were withdrawn for protein quantification and 0.3 mL of cold methanol (MeOH) were added. The samples were centrifuged at 1016×g for 15 min (4° C.) and the resulting supernatants were submitted to solid phase extraction of lipids using HLB plate (OASIS® HLB 30 mg, 96-well plate, Waters, Saint-Quentin-en-Yvelines, France). Briefly, plates were conditioned with 500 μL MeOH and 500 μL H2O/MeOH (90:10, v/v). Samples were loaded at a flow rate of about one drop per 2 s and, after complete loading, columns were washed with 500 μL H2O/MeOH (90:10, v/v). The phase was thereafter dried under aspiration and lipids were eluted with 750 μL MeOH. Solvent was evaporated under N2 and samples were resuspended with 140 μL MeOH and transferred into a vial (Macherey-Nagel, Hoerdt, France). Finally, the 140 μL of methanol were evaporated and our sample resuspended with 10 μL of methanol for liquid chromatography/mass spectrometry analysis. 6-keto-prostaglandin F1 alpha (6kPGF1α), thromboxane B2 (TxB2), Prostaglandin E2 (PGE2), 8-iso Prostaglandin A2 (8-isoPGA2), Prostaglandin E3 (PGE3), 15-Deoxy-□12,14-prostaglandin J2 (15d-PGJ2), Prostaglandin D2 (PGD2), Lipoxin A4 (LxA4), LxB4, Resolvin D1 (RvD1), Resolvin D2 (RvD2), Resolvin D5 (RvD5), 7-Maresin 1 (7-Mar1), Leukotriene B4 (LtB4), Leukotriene B5 (LtB5), Protectin Dx (PDx), 18-hydroxyeicosapentaenoic (18-HEPE), 5,6-dihydroxyeicosatetraenoic acid (5,6-DiHETE), 9-hydroxyoctadecadienoic acid (9-HODE), 13-hydroxyoctadecadienoic acid (13-HODE), 15-hydroxyeicosatetraenoic acid (15-HETE), 12-hydroxyeicosatetraenoic acid (12-HETE), 8-hydroxyeicosatetraenoic acid (8-HETE), 5-hydroxyeicosatetraenoic acid (5-HETE), 17-hydroxydocosahexaenoic acid (17-HDoHE), 14-hydroxydocosahexaenoic acid (14-HDoHE), 14,15-epoxyeicosatrienoic acid (14,15-EET), 11,12-epoxyeicosatrienoic acid (11,12-EET), 8,9-epoxyeicosatrienoic acid (8,9-EET), 5,6-epoxyeicosatrienoic acid (5,6-EET), 5-oxoeicosatetraenoic acid (5-oxoETE), Prostaglandin F2α, (PGF2α), 13-Hydroxyoctadecadienoic acid (13oxoODE), 9-hydroxyoctadecadienoic acid (9oxoODE), 10-hydroxyoctadecadienoic acid (10-HODE), 9,10-dihydroxy-12-octadecenoic acid (9,10-DiHOME), 12,13-dihydroxy-12-octadecenoic acid (12,13-DiHOME), 9-hydroxy-10,12,15-octadecatrienoic acid (9-HOTrE), 13-hydroxy-9,11,15-octadecatrienoic acid (13-HOTrE), 9,10,13-trihydroxy-11-octadecenoic acid (10-TriHOME) and 9,12,13-trihydroxy-11E-octadecenoic acid (12-TriHOME) were quantified in mouse colons. To simultaneously separate 42 lipids of interest and three deuterated internal standards (5-HETEd8, LxA4d4 and LtB4d4), LC-MS/MS analysis was performed on an ultrahigh-performance liquid chromatography system (UHPLC; Agilent LC1290 Infinity) coupled to an Agilent 6460 triple quadrupole MS (Agilent Technologies) equipped with electrospray ionization operating in negative mode. Reverse-phase UHPLC was performed using a Zorbax SB-C18 column (Agilent Technologies) with a gradient elution. The mobile phases consisted of water, acetonitrile (ACN), and formic acid (FA) [75:25:0.1 (v/v/v)](solution A) and ACN and FA [100:0.1 (v/v)](solution B). The linear gradient was as follows: 0% solution B at 0 min, 85% solution B at 8.5 min, 100% solution B at 9.5 min, 100% solution B at 10.5 min, and 0% solution B at 12 min. The flow rate was 0.4 ml/min. The autosampler was set at 5° C., and the injection volume was 5 μL. Data were acquired in multiple reaction monitoring (MRM) mode with optimized conditions. Peak detection, integration, and quantitative analysis were performed with MassHunter Quantitative analysis software (Agilent Technologies). Blank samples were evaluated, and their injection showed no interference (no peak detected), during the analysis. Hierarchical clustering was performed, and heat maps were obtained with R (www.rproject.org). PGJ2, RVD1, RVD2, RVD5, 7Mar1, 5,6-DiHETE were not detected in our samples. PUFA metabolite amounts were transformed to z scores and clustered based on 1-Pearson correlation coefficient as distance and the Ward algorithm as agglomeration criterion.

### Dosage of the Corticosterone

Mice were anesthetized with ketamine/xylasine and submandibular blood was collected into tubes and centrifuged (15 min, 8000 rpm) to separate the serum. Serum samples were stored at −80° C. until analysis. Corticosterone levels were determined using the corticosterone AlphaLISA detection kit (PerkinElmer, AL3020C) following manufacturer's instructions. The volumes of all reagents were adjusted for the use 5 μl of serum or standard. The serum samples were diluted 1:5 into 1× diluent. The corticosterone concentrations in serum samples were expressed in ng/ml. (n=8 to 10 mice/group).

### Phenotypic Analysis of Mesenteric Lymph Node Cells

11 weeks old controls or prenatally stressed mice were euthanized and their mesenteric lymph node cells were stained with conjugated antibodies for flow cytometry analysis. The monoclonal antibodies (mAbs) used for flow cytometry were as follows: anti-TCRαβ-BV711(H57-597), anti-CD4-BV786, (GK1.5), anti-CD8α-A700 (53-6.7), anti-CD44-Percep5.5 (IM7) and anti-CD62L-APC (MEL-14), and with a viability dye (APC-H7) to exclude dead cells. We used the Foxp3/transcription factor permeabilization kit according to the manufacturer's instructions (e-Biosciences) to fix and permeabilize the cells prior to intracellular staining with anti-Foxp3 labelled antibody (Foxp3-BV421 (FJK-16s)). The fluorescently conjugated antibodies were purchased from e-Biosciences, BD Biosciences, and Biolegend. Data were collected on LSR-Fortessa cytometer (BD Biosciences) and analyzed using the FlowJo software.

### Fecal Bacterial DNA Extraction

Feces were collected and frozen at −80° C. until use. 15 feces/group chosen from 3 independent experiments. Metagenomic DNA was extracted from the feces using a QIAamp DNA Stool Mini Kit (Qiagen, Hilden, Germany) according to the manufacturer's instructions. Quickly, the feces were crushed in the inhibitex buffer at (6.5 ms-1, 3×30 s), placed at 95° C. and then were centrifuged 2 min, at 16 000 g, at room temperature to pellet the stool particles. 200 μL of the supernatant were added to 15 μL of proteinase K and AL buffer and incubated 10 min at 70° C. 200 μL of EtOH were added and the samples were added in the spin columns and centrifuged at 16 000 g at room temperature for 1 minute. The supernatant was removed and 500 μL of washing buffer 1 was added. After centrifugation at 16 000 g at room temperature for 1 minute, the supernatant was removed and 500 μL of washing buffer 2 was added. After centrifugation at 16 000 g at room temperature for 3 minutes, the supernatant was removed and 50 μL of the elution buffer was added. Finally, the samples were centrifuged for 1 minute at 16 000 g at room temperature and the DNA was collected. The DNA was then quantified using a Nanodrop.

### Taxonomic and Predicted Functional Analysis of Gut Microbiota

Total DNA was extracted from freshly-collected snap-frozen feces as already described [S. Nicolas et al., «Transfer of dysbiotic gut microbiota has beneficial effects on host liver metabolism», Mol. Syst. Biol., vol. 13, no 3, p. 921, mars 2017, doi: 10.15252/msb.20167356.]. The 16S rRNA gene V3-V4 regions were targeted by the 357wf-785R primers and analyzed by MiSeq at RTLGenomics (http://rtlgenomics.com/, Texas, USA). An average of 29795 sequences was generated per sample. A complete description of the applied bioinformatic filters is available (http://www.rtlgenomics.com/docs/Data_Analysis_Methodology.pdf). LDA scores were drawn by the Huttenhower Galaxy web application (http://huttenhower.sph.harvard.edu/galaxy/) via the LEfSe algorithm [N. Segata et al., «Metagenomic biomarker discovery and explanation», Genome Biol., vol. 12, no 6, p. R60, juin 2011, doi: 10.1186/gb-2011-12-6-r60.]. The predictive functional analysis (gut microbiome) of gut microbiota was performed via PICRUSt [M. G. I. Langille et al., «Predictive functional profiling of microbial communities using 16S rRNA marker gene sequences», Nat. Biotechnol., vol. 31, no 9, p. 814-821, sept. 2013, doi: 10.1038/nbt.2676.]. Diversity indices were calculated with the software PAST4 [O. Hammer, D. A. T. Harper, et P. D. Ryan, «PAST: Paleontological Statistics Software Package for Education and Data Analysis», p. 9.].

### Biofilm

For fluorescent in situ hybridization, a specimen of colon with a feces was resected, fixed in carnoy (60% MeOH, 30% chloroform, 10% acetic acid), embedded in paraffin, sectioned, and labelled with the universal bacterial 16 S fluorescent rRNA probe EUB338-Cy3, 5′-GCTGCCTCCCGTAGGAGT-3′ Cy5, Eurofins) at 10 μL/mL, wheat germ agglutinin-FITC 1/1000 (Sigma-Aldrich L4895) was used to stain the polysaccharide-rich mucus layer, and the epithelial cell nucleus was stained with DAPI (ProLong Gold antifade reagent with DAPI, Invitrogen P36935). Bacterial penetration into the mucus was measured by image processing on Fiji by quantifying the number of 16S RNA labelled pixel between the edge of the lumen to the edge of the epithelium (n=12 mice/group; 4 images/mice, 2 independent experiments). The mucus zone was manually traced, and an arbitrary distance was assigned to each pixel from the middle to the epithelial cells border. Cyanine 5 labelled pixels in this zone were assigned to their corresponding distance. The distance of each pixel in regard to the luminal compartment and the number of labeled pixels between the edge of the lumen to middle of the mucus (Apical) and between the middle of the mucus to the edge of the epithelium (Basal) were quantified.

### Identification of Colonic Mouse Bacteria

Ligilactobacillus murinus strain IRSD_2020, Limosilactobacillus reuteri and Lactobacillus johnsonii/gasseri were isolated from mouse intestinal mucosa on Lactobacillus selection agar (BD Diagnostic, Le Pont de Claix, France), and identified by matrix-assisted laser desorption/ionisation time-of-flight mass spectrometry (Microflex LT MALDI-TOF MS, Bruker Daltonik GmbH, Germany). Whole genome sequencing of Ligilactobacillus murinus strain IRSD_2020 has been performed as well as the quantification of this strain and of Ligilactobacillus animalis by Tagman® real-time PCR. Ligilactobacillus murinus strain IRSD_2020 was cultured and GABA-containing lipopeptides quantified.

Whole Genome Sequencing of Ligilactobacillus murinus IRSD_2020

Genomic DNA from L. murinus IRSD_2020 and L. animalis DSMZ 20602 were purified from 200 μL overnight cultures with MagNA Pure 96 DNA and Viral NA Small Volume Kit (Roche Diagnostics France SAS, Meylan, France) and sequenced in 2×150 bp paired-end by Illumina® NextSeq500 (IntegraGen SA, Evry, France) with an 80× coverage. Libraries were obtained by enzymatic fragmentation using a 5×WGS Fragmentation mix kit (Enzymatics Inc., Beverly, MA, USA). The sequence data generated were deposited in the NCBI BioProject database (https://www.ncbi.nlm.nih.gov/bioproject/) under the accession the number PRJNA770189.

Quantification of Ligilactobacillus murinus IRSD_2020 and Ligilactobacillus Animalis Using TaqMan® Real-Time PCR

Primers and probes included in the real-time PCR assay targeting L. murinus were selected in an 834 bp-long open reading frame (ORF) whose sequence was retrieved from L. murinus IRSD_2020 genome and was found with >99% similarity in other L. murinus strains and with <77% similarity in Streptococcus suis (data not shown). Primers and probes included in the real-time PCR assay targeting L. animalis were selected in the 16S-23S internal transcribed spacer (ITS) region. All the primers and probes were designed with TaqMan® Primer and Probes Design Tool (Genescript, Leiden, the Netherlands) and purchased from Eurofins Genomics (Germany). Detection was achieved with LightCycler480II (Roche Diagnostics, Meylan, France) real-time PCR system according to the manufacturer's instructions. TaqMan® qPCRs were performed in 10 μL reactions containing: 1× iQ Supermix (Biorad), 0.3 μM each primer, 0.2 μM probe, 2 μl purified DNA diluted 1:10 and nuclease-free water. The bacterial DNA qPCR was performed using the following conditions: 95° C. for 3 min, followed by 45 cycles consisting of 95° C. for 15 s and 600 for 60 s. Duplicate reactions were run for all samples. The cycle threshold of each sample was then compared with a DNA standard. DNA standard was obtained by purification of Ligilactobacillus genomic DNA from the 10 ml pellet of overnight culture using the QIAamp Fast DNA Stool Mini Kit (Qiagen, Hilden, Germany). Ten-fold serial dilutions of DNA standard were used to generate a standard curve. The data was expressed as Ligilactobacillus copies per ng of isolated DNA.

### Bacterial Culture

L. murinus IRSD_2020 was grown on Lactobacilli MRS broth (Difco, Fisher scientific SAS, Illkirch, France)—agar plates supplemented with cysteine chloride monohydrate 0.5 g·L-1. After 24 hours of incubation at 37° C., 3 colonies were seeded in 30 mL of MRS broth supplemented with cysteine chloride monohydrate 0.5 g·L-1 (Merck-Sigma Aldrich, St. Quentin Fallavier, France) and incubated overnight at 37° C. Cultures have been performed with or without GABA (8 mg/mL; Sigma A2129, St. Quentin Fallavier, France) in the medium. All the culture steps have been performed in a hypoxic environment ensured by using a Whitley H35hypoxystation (don Whitley scientific, Bingley, United Kingdom) and all the incubations were done in an anaeropack jar (Fisher scientific SAS, Illkirch, France) with an anaeroGen sachet (Fisher scientific SAS, Illkirch, France) to ensure an anaerobic environment. Lipopeptides containing GABA were quantified in these bacteria.

### Bacterial Lipopeptide Quantification

To quantify bacterial lipopeptides, mass-spectrometry method has been adapted from the C12AsnGABA quantification method previously described [T. Perez-Berezo et al., «Identification of an analgesic lipopeptide produced by the probiotic Escherichia coli strain Nissle», Nat. Commun., vol. 8, no 1, December 2017, doi: 10.1038/s41467-017-01403-9.] following our previous manuscript on the mass-spectrometry analyses of aminolipids and lipopeptides [A. Hueber et al., «Identification of bacterial lipo-amino acids: origin of regenerated fatty acid carboxylate from dissociation of lipo-glutamate anion», Amino Acids, vol. 54, no 2, p. 241-250, févr. 2022, doi: 10.1007/s00726-021-03109-1.], [A. Hueber et al., «Discovery and quantification of lipoamino acids in bacteria», Anal. Chim. Acta, vol. 1193, p. 339316, févr. 2022, doi: 10.1016/j.aca.2021.339316.]. For the extraction, 500 μL of Tris buffer (pH=9), 1 mL of MeOH and 5 μL of IS*C16AsnGABA were added to the bacterial pellets or colon samples and crushed with a Fast Prep instrument (MP Biomedicals), using two cycles (6.5 m·s-1, 30 s). For the human feces, 300 mg of feces were crushed in 500 μL of Tris buffer (pH=9) and 1 mL of MeOH as described above. In ⅓ of the extract (500 μL), 5 μL of IS*C16AsnGABA and 1 mL of Tris/MeOH were added. Ten μL of suspension were withdraw for protein quantification with Biorad assays, then, 6.6 mL of water were added to the homogenate. Samples were centrifuged at 1016×g for 15 min (4° C.) and the supernatants submitted to SPE using HLB plates (HLB, 30 mg, Waters). Plates were conditioned with 750 μL ethyl acetate, 750 μL MeOH and 750 μL H2O:MeOH (90:10; v/v). Samples were loaded at a flow rate of one drop per 2 s and SPE plates washed using 1 mL H2O:MeOH (90:10; v/v) followed by 1 mL heptane. Lipopeptides were eluted using 1 mL AcN, 1 mL MeOH and 1 mL AcOEt. Eluent were carefully removed from the plate, and transferred to a Pyrex tube, dried under nitrogen gas and reconstituted in 10 μL MeOH for LC-QqQ (ou LC-LRMS) analysis. Extract was stored at −20° C. before LC-MS analysis. High-performance liquid chromatography system was a Shimadzu Mikros LC system, equipped with a thermostated autosampler SIL-30AC, a rack changer II, a Nexera Mikros binary pump, a degasser on-line DGU-20A3R and an LTO-Mikros column oven. The analytical column was a CSH C18 column Waters (1×100 mm; 2.7 μm) and was maintained at 40° C. The mobile phases consisted of water:FA (99.9:0.1; v/v) (A) and acetonitrile:FA (99.9:0.1, v/v) (B). Flow rate was 0.1 mL·min-1. The multi-step gradient starts with 30% B at 0 min, 80% B at 13 min, 100% B at 13.5 min, 100% B at 16 min, and at 16.5 min 30% B until 18 min. The chromatography system was coupled online to a triple quadrupole mass spectrometer Shimadzu 8060 equipped with an ESI source in negative mode and was optimized as follow: nebulizer: 2 L·min-1; desolvation line: 250° C.; heating-block: 450° C., heating gas flow: 10 L·min-1, and no drying gas was used. Argon gas (purity, >99.9995%) was used for collision-induced dissociation (CID). For each metabolite the MRM transition (Table S5) was optimized on the pure standard to get the best selectivity and the best sensitivity with a qualitative and a quantitative transition and was programmed to monitor a 2 min-window (expected chromatographic retention time 1 min). The dwelltime were optimized for each compound and were set between 15 and 100 msec. Peak detection, integration, and quantitative analysis were performed with LabSolution software (Version 5.99 SP2), Shimadzu.

### Calcium Imaging of Sensory Neurons

Mouse dorsal root ganglia were dissociated as previously described [N. Cenac et al., «Potentiation of TRPV4 signaling by histamine and serotonin: an important mechanism for visceral hypersensitivity», Gut, vol. 59, no 4, p. 481-488, 2010, doi: 10.1136/gut.2009.192567.]. After 48 h of culture, cells were incubated with HBSS containing 20 mM HEPES, 1 mM fluo-4 acetoxymethyl (AM) and 20% pluronic F-127 for 30 min at 37° C. plus 30 min in the dark at RT. The plates were then washed with HBSS and 140 μl of HBSS were added to each well. Neurons were pre-treated with C16LeuGABA (0.1, 1, and 10 μM), or vehicle (HBSS/MeOH 0.06%) for 5 min and then were stimulated with either capsaicin or the mix of GPCR agonists. Live cell imaging of calcium was carried out on an automated inverted microscope (Axio Observer, Zeiss, SAS France) with a ×10 objective (NA 0.45). Images were acquired using the Zen imaging software (3.1 Blue Edition) and a kinetic of 85 recordings (one per second) was performed. The first ten images were used to determine the baseline and, from 10 to 60 sec, neurons were exposed to either a mix of GPCR agonists (histamine, bradykinin, serotonin, 10 μM), capsaicin (500 nM) or vehicle (HBSS). After 60 seconds, neurons were treated with KCl (50 mM) in order to discriminate neurons from glial cells. The ImageJ software was used to perform the analysis of calcium flux.

### Statistical Analysis

To perform a multivariate analysis of our cohort, the data obtained from 56 mice (4 groups with 14/group) from 3 different experiments were used to create a matrix including different variables: gene expression, PUFA quantification, VMR to colorectal distension, faecal bacteria abundance from the 16S RNA sequencing, and colon thickness. Principal Component Analysis (PCA) were performed to detect outliers and intrinsic clusters. Then PLS-DA was applied to study the relationship between prenatal stress exposure and variables described above. From this analysis, discriminant variables (with a value of Variance Importance in the Projection, VIP, >1.0) were selected. Wilcoxon test with FDR correction was applied on selected variables. PCA, PLS-DA and Wilcoxon test were done using the Galaxy Workflow 4 metabolomics (W4m) instance [E. A. Thevenot, A. Roux, Y. Xu, E. Ezan, et C. Junot, «Analysis of the Human Adult Urinary Metabolome Variations with Age, Body Mass Index, and Gender by Implementing a Comprehensive Workflow for Univariate and OPLS Statistical Analyses», J. Proteome Res., vol. 14, no 8, p. 3322-3335, août 2015, doi: 10.1021/acs.jproteome.5b00354.], [Y. Guitton et al., «Create, run, share, publish, and reference your LC-MS, FIA-MS, GC-MS, and NMR data analysis workflows with the Workflow4Metabolomics 3.0 Galaxy online infrastructure for metabolomics», Int. J. Biochem. Cell Biol., vol. 93, p. 89-101, December 2017, doi: 10.1016/j.biocel.2017.07.002.]. Correlations were performed by Spearman test. Data are presented as means±standard error of the mean (SEM). Analyses were performed using GraphPad Prism 9.0 software (GraphPad, San Diego, CA). Due to the small sample size and scoring, comparisons between groups were mainly performed by Mann-Whitney non-parametric test. Multiple comparisons within groups were performed by Kruskal-Wallis test, followed by Dunn's post-test. Parametric two-way Anova was used when data were linearly distributed and followed by Bonferroni posthoc test. Statistical significance was set at P<0.05.

### Results

### Prenatal Stress Induces Visceral Hypersensitivity in the Adult Offspring

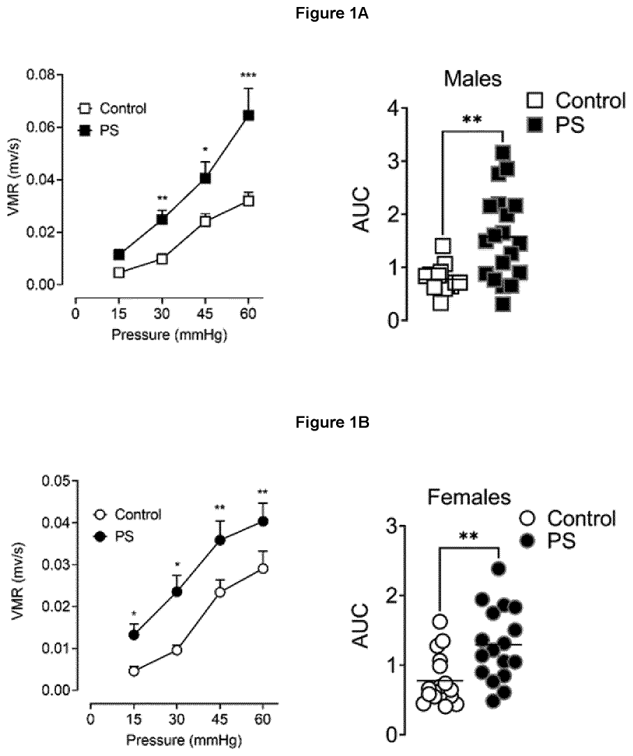

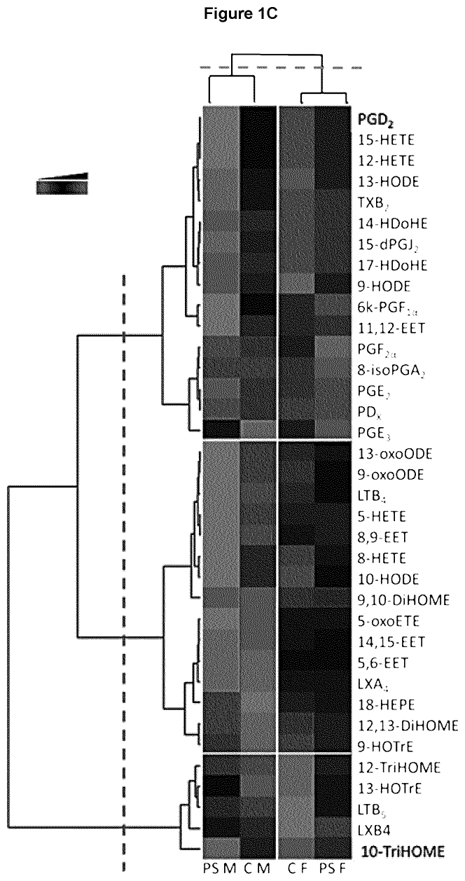

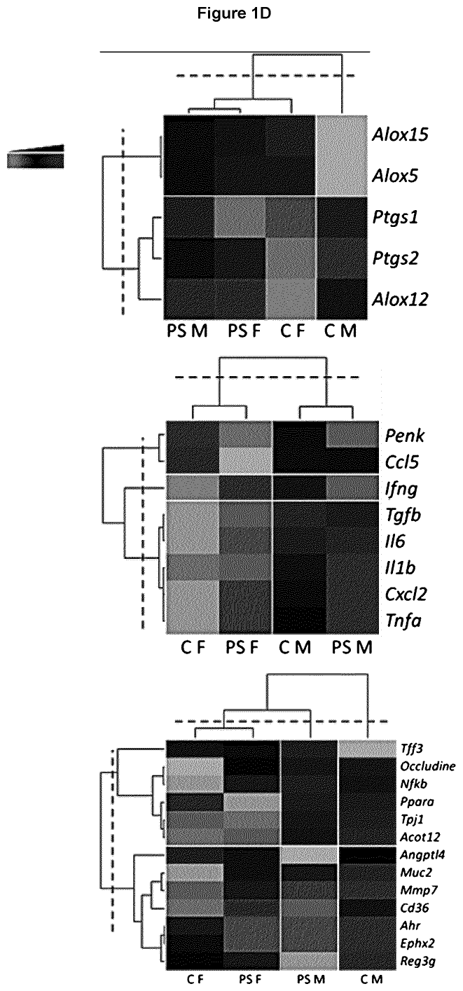

We measured the impact of PS on visceral sensitivity in adulthood by measuring visceromotor responses (VMR) to colorectal distension (CRD). In PS male and female offspring, the VMR and the AUC of the VMR was significantly increased compared to control mice (FIG. 1A and FIG. 1i). No significant differences in visceral paracellular permeability, colon thickness and inflammatory scores at the macroscopic and microscopic level were observed in offspring from stressed dams as compared to controls. PS altered neither basal plasma corticosterone nor the expression of corticosterone receptors in the colon of the adult offspring (data not shown).

At the molecular level, polyunsaturated fatty acid (PUFA) metabolites were quantified in the mouse colons using liquid chromatography coupled to mass spectrometry (LC-MS/MS). Mice hierarchical clustering showed that the mice were not separated by their stress status but by their sex (FIG. 1C) demonstrating that PS had little or no impact on colonic PUFA metabolism. PUFA metabolites hierarchical clustering revealed three different clusters (FIG. 1C). The first one regrouped products derived from the metabolism of PUFAs by cyclooxygenases (COX) (PGE2, TXB2, 6k-PGF1α, PGF20, PGD2, 8-isoPGA2, 15-dPGJ2 and PGE3), by lipoxygenases (LOX) (15-HETE, 12-HETE, 9-HODE, 13-HODE, PDx, and 14-HDoHE) and by cytochromes epoxygenases (11,12-EET). The means of these metabolites' concentrations were higher in females than in males, but no statistical differences were observed between PS and control mice, except for the 11,12-EET, which was significantly decreased in PS male vs control (data not shown). The second cluster was composed of LOX metabolites (13-oxoODE, 9-oxoODE, 9-HOTrE, LTB4, LXA4, 5-HETE, 5-oxoETE, 8-HETE, and 18-HEPA), CYP metabolites (8,9-EET, 9,10-DiHOME, 14,15-EET, 5,6-EET, 12,13-DiHOME) and by the 10-HODE. In this cluster, the concentration of 5-oxoETE, 5-HETE, 8-HETE, LXA4, 10-HODE, 8,9-EET and 14-15-EET was significantly decreased in PS male offspring compared to control mice (data not shown). Other PUFA metabolites from the LOX pathway (10-TriHOME, 12-TriHOME, LTB5, LXB4, and 13-HOTrE) were portrayed in the last cluster and their mean concentrations were increased in males compared with females, but no statistical differences were observed (data not shown). In addition, we quantified mRNA expression of genes coding for the main enzymes implicated in PUFA metabolism. Heatmap of the expression of PUFA metabolizing enzymes showed a hierarchical separation of the control males from the three other experimental groups. 5-lipoxygenase and 15-lipoxygenase expression clustered apart form 12-lipoxygenase, Cox-1 and Cox-2 mRNA expression. Only Alox5, which codes for the 5-lipoxygenase was increased in PS males compared to control (data not shown). PS increases neither pro-inflammatory lipid mediators nor the enzymes implicated in their synthesis, with the exception of the 5-lipoxygenase, in adult male mice.

Then, we assessed the mRNA expression of genes implicated in colonic immune response and homeostasis (FIG. 1D). Again, hierarchical clustering showed that mice were not separated by their stress status but by their sex and that genes were clustered in three groups Penk and Ccl5 (cluster 1), Ifng (cluster 2) and Tgfb, Il6, Il1b, Cxcl2 and Tnfa (cluster 3). No statistical differences were observed between PS and control adult male and female mice. In agreement, the percentages of CD8+ and CD4+ T cells, regardless of their conventional or memory phenotype, were not significantly different between control and PS mice (data not shown). The expression of genes significant for the epithelium homeostasis grouped the female mice together, unlike males that were separated by the hierarchical clustering depending on their stress status. Control male mice were represented by an increase in the means of the expression of Ocln, Nfkb, Ppara, Tjp1, Acot12, Angptl4, Muc2, Mmp7, Cd36, Ahr, Ephx2 and Reg3g, whereas PS males were represented by an increase of Tff3, Ocln, Njkb, Ppara, Tjp1 and Acot12 (FIG. 1D). In female mice, the means of expression of these genes were lower than in male mice. No statistical difference between control and PS mice were observed (data not shown). In conclusion, prenatal stress induced visceral hypersensitivity in the absence of any sign of colonic inflammation in adulthood.

### Prenatal Stress Induces a Gut Microbiota Dysbiosis and Alters Gut Microbiota Spatial Organization in Adulthood.

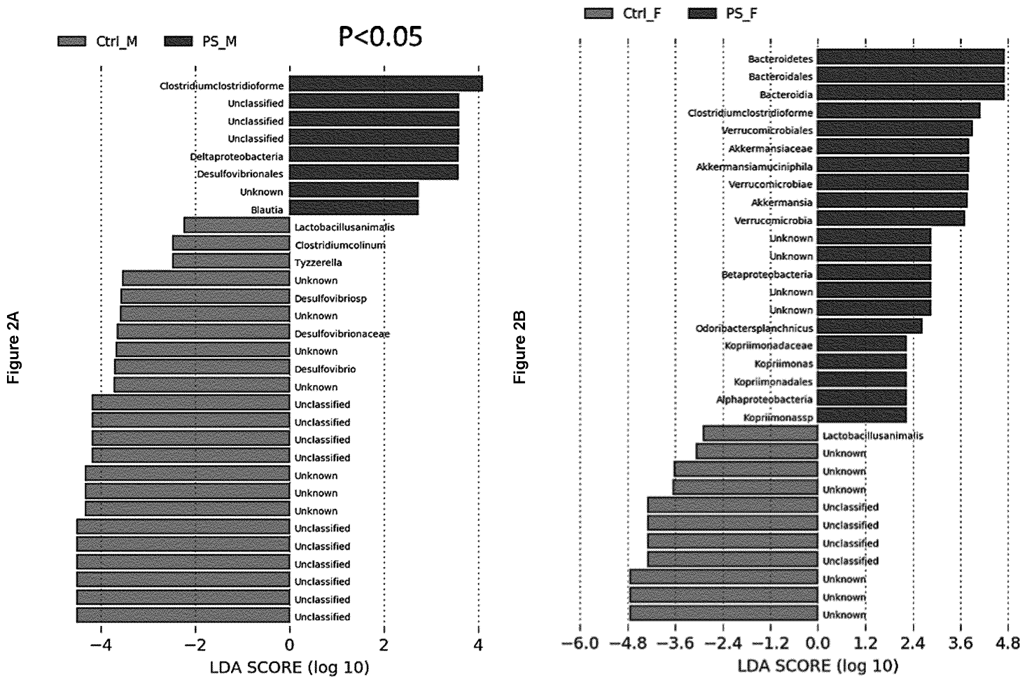

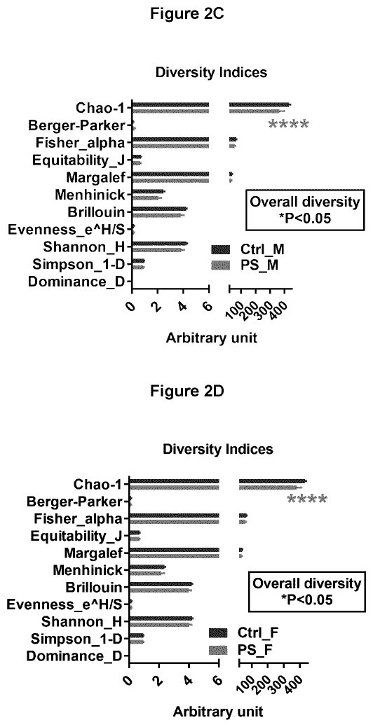

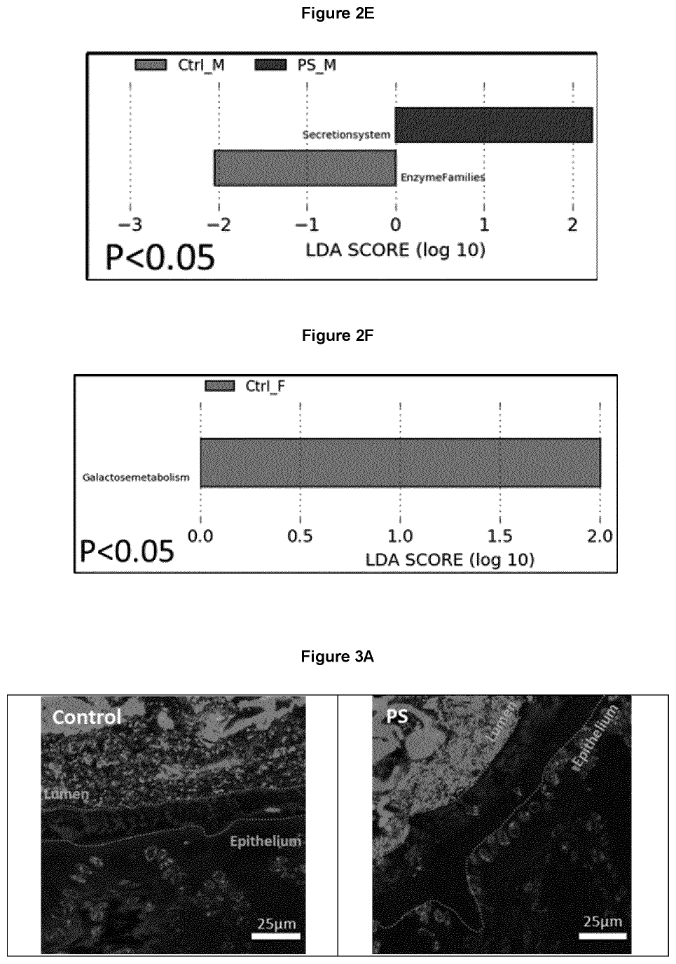

The 16S RNA analyses of the feces revealed a shift in the gut microbiota composition in PS mice that differed in male and female offspring. In males, PS mice showed a higher relative abundance of Clostridium clostridioforme, Deltaproteobacteria, Desulfovibrionales and Blautia, while the relative abundance of Desulfovibrio and Desulfovibrionaceae as well as Tyzzerella and Clostridium colinum was higher in control mice (FIG. 2A). In females, the relative abundances of taxa from Bacteroidetes and Verrucomicrobia phyla were higher than in control mice (FIG. 2B). In both PS male and female offspring, the relative abundance of Clostridium clostridioforme was higher, whereas the relative abundance of Lactobacillus animalis was lower (FIGS. 2A and 2B). These taxonomical differences were in accordance with a significant different and lower overall diversity, mostly based on Chao-1 index, related to rare species, for both PS male and female mice (FIGS. 2C and 2D). We also performed a PICRUSt-based predictive analysis of gut microbial functions. In males, PS mice showed a higher function related to secretion system whereas control mice displayed higher undefined enzyme families (FIG. 2E). In females, the predictive gut microbiome did not vary compared to control mice, which showed a higher galactose metabolism (FIG. 2F).

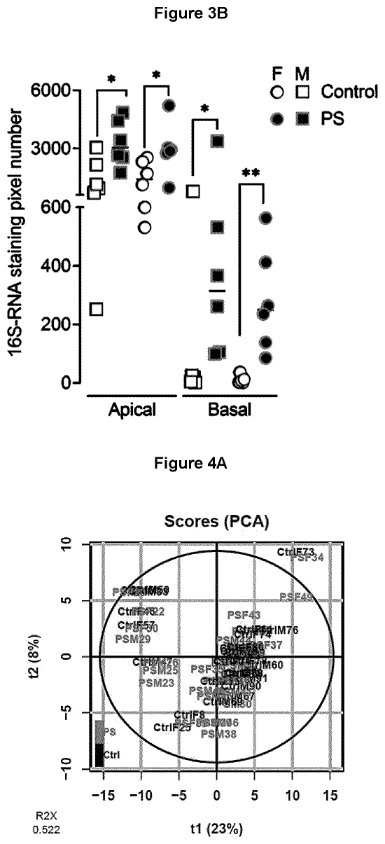

Beyond taxonomic and functional alteration of the gut microbiota, PS induced a spatial organizational change of the colonic microbiota. In control mice of either sex, bacteria were separated from the epithelial cells by a dense sterile mucus layer (FIG. 3). In contrast, in PS mice, some bacteria from the gut lumen invaded this layer (FIG. 3). By quantifying the 16S RNA labelled pixels inside the mucus layer, we showed that in PS mice, more bacteria crossed the border with the lumen and moved close to the epithelium (FIG. 3).

Overall, these data show that prenatal stress induces gut microbiota dysbiosis and biogeographical changes in adulthood.

### The Abundance of L. Animalis is Inversely Correlated to Visceral Hypersensitivity

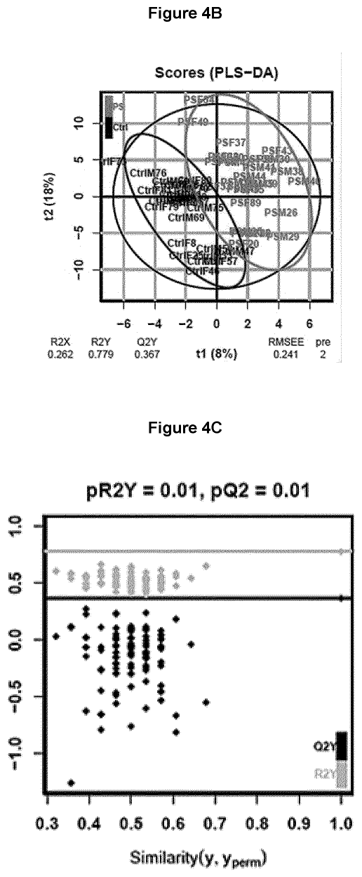

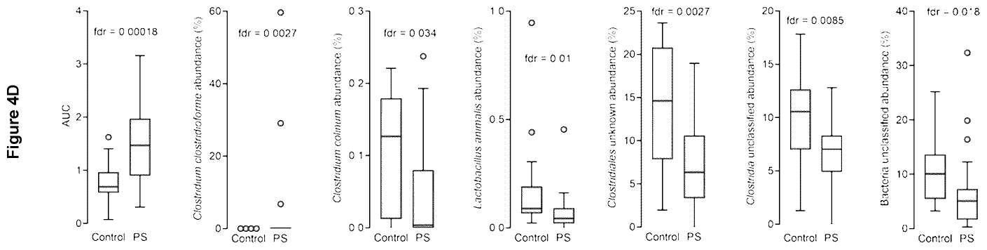

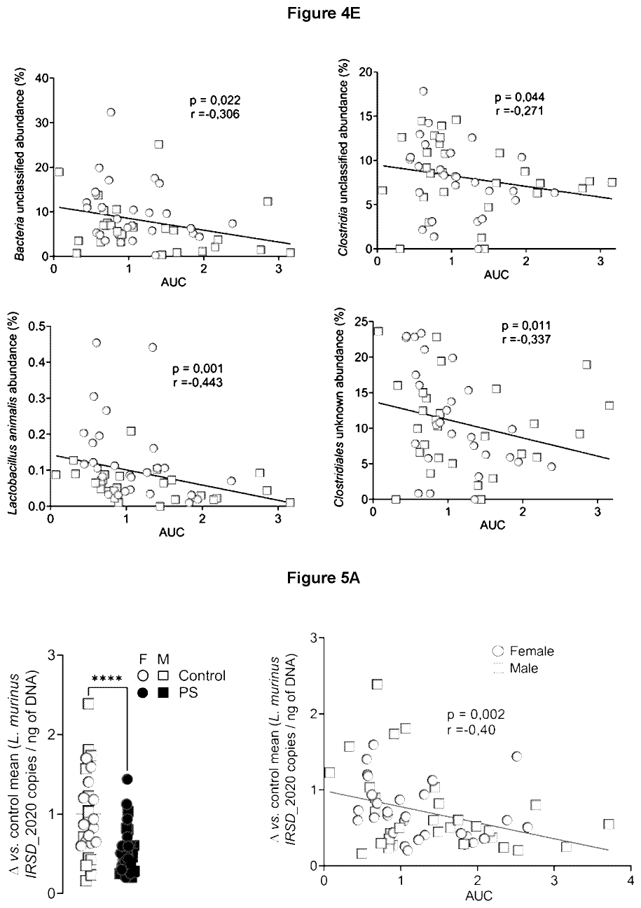

To extract the important differences between prenatally stressed and control mice, we first ran a PCA model followed by a PLS-DA model. In both models, the mice were mainly located in within the 95% confidence intervals (black ellipse) (FIGS. 4A and 4B). The plotted PCA scores showed the aggregation and dispersion of the mice, and no outliers were identified (FIG. 4A). The PLS-DA scores plot was fitted on Unit variance-scaled data and displays the classification effect. It showed a clear discrimination between the control and PS groups (FIG. 4B) and the underlying model was validated by a robust (p<0.05) permutation test (FIG. 4C). Seven variables had a variable importance in projection (VIP) value≥1 and a p-value≤0.05, showing their importance for group separation in the PLS-DA model. Besides visceral sensitivity (AUC), six bacterial species were identified as important: Clostridia unclassified, Lactobacillus animalis, Clostridium clostridioforme, Clostridium colinum, Clostridiales unknown, Bacteria unclassified (FIG. 4D). However, when similar analyses were done on each sex separately, visceral sensitivity was a common variable for separating Ctrl and PS mice, while significant bacteria abundances were different in male and female. We performed Spearman correlations between these variables to consolidate the link between the identified bacterial species and visceral hypersensitivity. Clostridia unclassified, Lactobacillus animalis, Clostridiales unknown and Bacteria unclassified were inversely correlated to visceral sensitivity (FIG. 4E). The highest correlation coefficient was reached with Lactobacillus animalis (FIG. 4E). In conclusion, visceral hypersensitivity and microbiota composition were the main drivers of the separation between control and PS mice and visceral hypersensitivity strongly correlated to the decreased abundance of L. animalis.

L. murinus Concentration is Decreased in Adult PS Feces

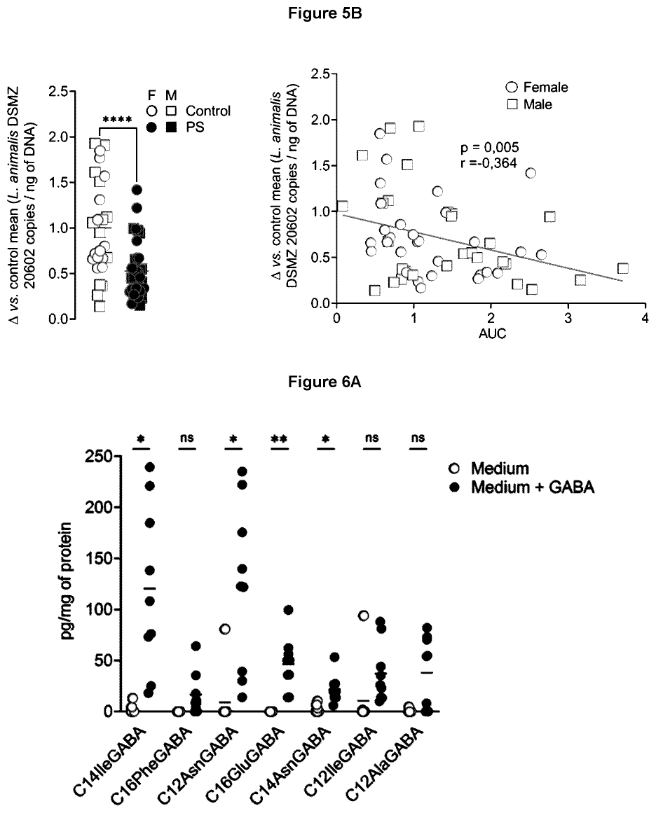

In the mucus of control but not PS mice, we identified by Maldi Tof the presence of Lactobacillus murinus, Lactobacillus reuteri and Lactobacillus johnsonii/gasseri. In 2020, the Lactobacillus genera regrouped 261 species very diverse from each other on the phenotypic, ecological and genotypic levels. Zheng et al. re-evaluated the taxonomy of Lactobacillaceae based on whole-genome sequencing and reclassified them into 25 genera [22]. On this basis, Lactobacillus reuteri is now classified as Limosilactobacillus reuteri and Lactobacillus murinus as Ligilactobacillus murinus. The latter is closely related to another species, Ligilactobacillus animalis and differentiating them from one another remains an issue using the current 16S RNA sequencing pipelines [23]. The same observation is done for probe designing as it is difficult to find species-specific 16S RNA locations. Therefore, it is now recommended to identify them as a single species named L. murinus animalis. As L. murinus animalis was the only identified bacterial species to be correlated with visceral sensitivity, we focused our attention on the role of this bacterium in intestinal homeostasis. Whole genome sequencing of Ligilactobacillus murinus, identified in the mice and named strain IRSD 2020, allowed us to determine that it possessed few sequences different from the reference strain DSMZ-20602 (data not shown). We then quantified L. murinus IRSD_2020 from mice fecal samples by Tagman® real-time PCR. L. murinus IRSD_2020 quantity was lower in the feces of PS mice in adulthood and was inversely correlated to the AUC of the visceral sensitivity (FIG. 5A). The same results were obtained with primers and probe designed for the L. animalis strain DSMZ-20602 (FIG. 5B).

L. murinus Produces Analgesic Lipopeptides

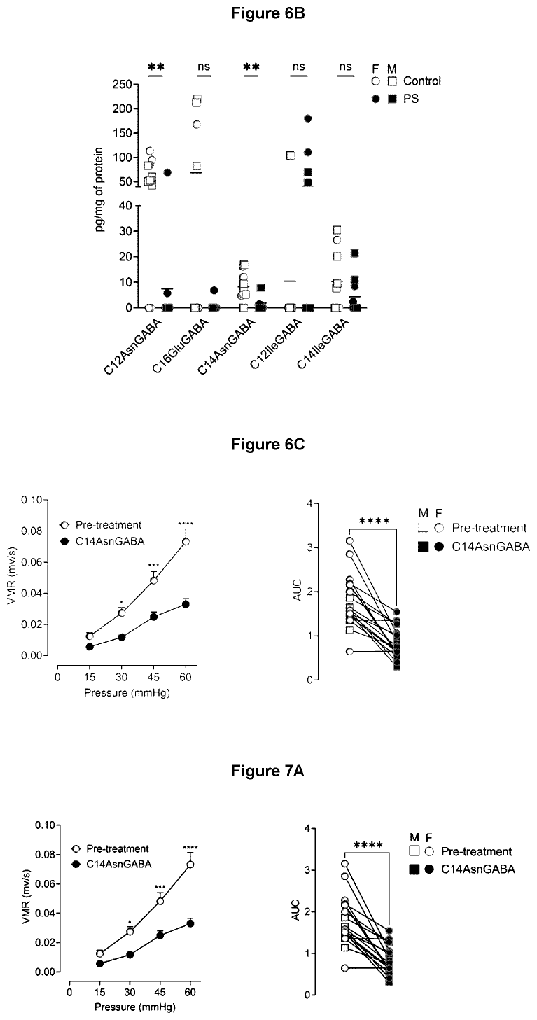

As the abundance of L. murinus TRSD_2020 was inversely correlated to the visceral sensitivity, we hypothesized that these bacteria produced a metabolite decreasing visceral sensitivity. Based on our previous study where we described the production of an analgesic GABA-containing lipopeptide by Escherichia coli strain Nissle 1917 [14] and on our studies identifying several lipoamines in bacteria [15,16], we developed a method to quantify the concentration of lipopeptides containing GABA: C12AlaGABA, C12AsnGABA, C14AsnGABA, C14:1AlaGABA, C12ValGABA, C12LeuGABA, C14GABA, C12IleGABA, C14IleGABA, C16LeuGABA, C16PheGABA and C16GluGABA (supplementary methods). In L. murinus IRSD 2020. as no clear production of lipopeptides-GABA was observed (FIGS. 6A & 8) and as these bacteria did not express the enzymes implicated in GABA synthesis, we cultured L. murinus IRSD_2020 in the presence of GABA. Under this condition, L. murinus IRSD_2020 produced several lipopeptides containing GABA: C16PheGABA, C12AsnGABA, C16GluGABA, C14AsnGABA, C12IleGABA, C14IleGABA and C12AlaGABA (FIG. 6A & FIG. 8). C14:1AlaGABA, C12ValGABA, C12LeuGABA, C14GABA and C16LeuGABA were not quantifiable in L. murinus IRSD_2020. The concentration of C12AsnGABA, C16GluGABA, C14AsnGABA, and C14IleGABA were significantly increased in bacteria cultured with 4 mg/ml of GABA (FIG. 6A). In mouse colons, we quantified C12AsnGABA, C16GluGABA, C14AsnGABA, C12IleGABA and C14IleGABA (FIG. 6B). C16PheGABA, C12AlaGABA, C14:1AlaGABA, C12ValGABA, C12LeuGABA, C14GABA and C16LeuGABA were not quantifiable in mouse colons. In the colon of PS mice, the concentration of C12AsnGABA and C14AsnGABA were significantly decreased compared to control (FIG. 6B). Finally, in a last set of experiments, CRD experiments were performed before and 30 minutes after intracolonic administration of 10 μM, as already described for the C12AsnGABA [14], of C14AsnGABA in male and female adult PS mice. Treatment by the lipopeptide decreased the visceral hypersensitivity induced by PS (FIG. 6C). We identified that L. murinus IRSD_2020 produced lipopeptides containing GABA in the presence of GABA. The treatment of sensitive mouse by C14AsnGABA, whose concentration was decreased in their colon, restored a normal visceral sensitivity.

Concentration of Neuronal Activation Inhibitory Lipopeptides is Decreased in Feces of Patients with IBS

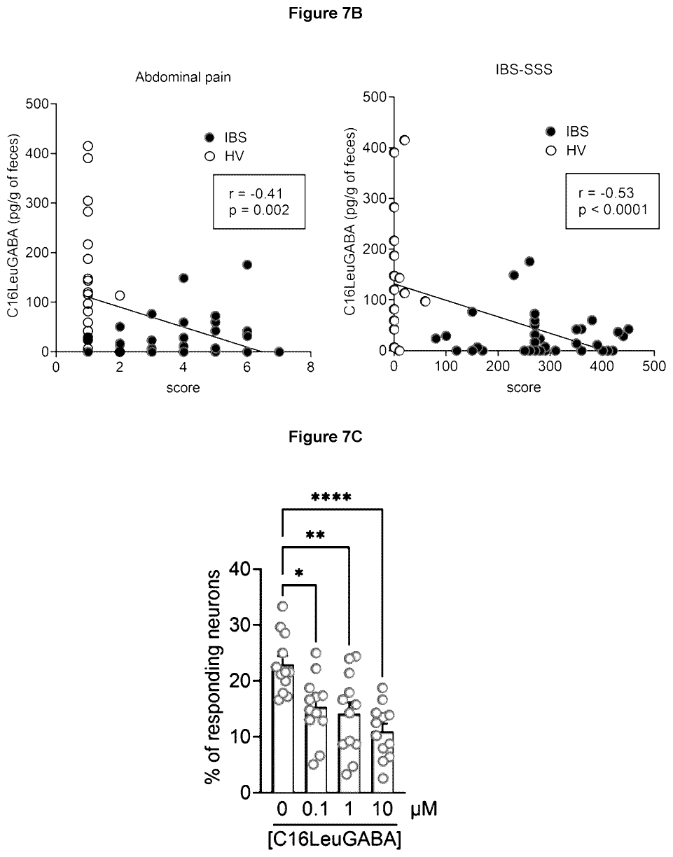

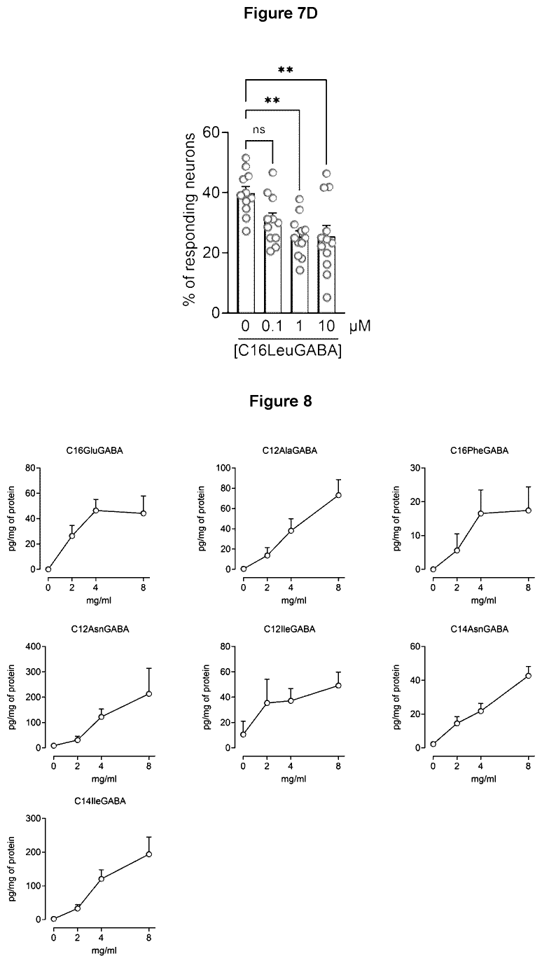

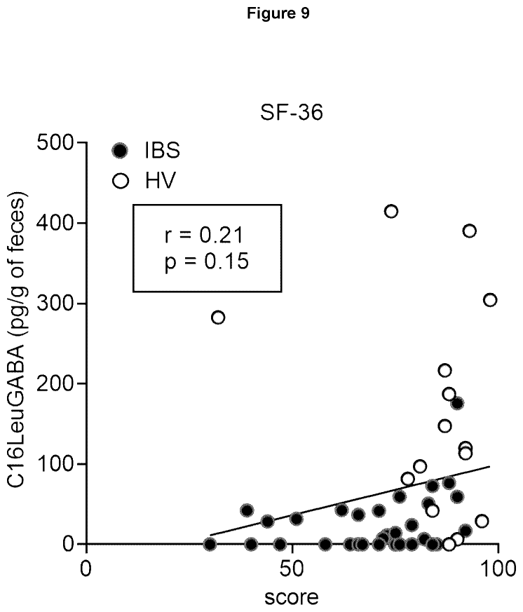

GABA-lipopeptides were quantified in feces from IBS patients and healthy volunteers using liquid chromatography-tandem mass spectrometry (LC-MS/MS). In contrast to L. murinus supplemented with GABA and mouse colon, only the C12AsnGABA and the C16LeuGABA were quantifiable in human feces. C12AsnGABA was quantifiable in 8 out of 18 healthy volunteers and was significantly decreased in patients with IBS (FIG. 7A). C16LeuGABA was quantifiable in 17 out of 18 healthy volunteers and was significantly decreased in patients with IBS (FIG. 7A). The concentration of C16LeuGABA was significantly inversely correlated to the IBS-SSS and to the abdominal pain score (FIG. 7B) and did not correlate with SF-36 (FIG. 9). As C16LeuGABA has never been quantified before, its role on sensory nerves activation was unknown. We next determined whether C16LeuGABA decreases neuronal activation, as previously described for C12AsnGABA [14]. Primary cultures of mouse dorsal root ganglia (DRG) neurons, activated either by an agonist of the receptor calcium channel TRPV1 (capsaicin) or by a mix of agonists (histamine, serotonin, and bradykinin) for G-protein-coupled receptors (GPCR), were treated with C16LeuGABA. Neurons exposure to either capsaicin (500 nM) or the GPCR agonists mix (histamine, bradykinin, serotonin, 10 μM each) induced an increase in calcium flux as shown by the higher % of responding neurons (FIG. 7C & FIG. 7D). The calcium flux increase induced by both nociceptive stimuli was prevented by C16LeuGABA pretreatment in a dose-dependent manner (FIG. 7C & FIG. 7D).

### Discussion

The onset of IBS in adulthood is associated with a higher number of early life adverse events [8]. However, due to the lack of data on prenatal period in humans, it is difficult to establish the causal link between stressful events during pregnancy and intestinal homeostasis disruption in adulthood. Here, in a mouse model of prenatal stress (PS), we show that the male and female offspring of stressed mothers present the main characteristics of IBS in adulthood: visceral hypersensitivity, no overt colonic inflammation, and a gut microbiota dysbiosis. Our results confirmed previous observations made in a rat model of prenatal stress where an increase in visceral sensitivity and no change of paracellular permeability have been described [24,25]. In contrast to stress performed in adulthood [26], we did not observe an increase in plasma corticosterone concentration in PS mice as previously observed in PS rodents [24, 25, 27, 28]. So, corticosterone is probably not implicated in the increase in visceral sensitivity observed in this model at adulthood. Nevertheless, we cannot exclude a long-lasting impact of corticosterone during foetal development and infancy. The gut microbiota of PS mice was however significantly altered in in both male and female offspring, characterized by an increased abundance of Clostridium clostridioforme and a decreased abundance of L. murinus animalis. Clostridium clostridioforme, reclassified as Enterocloster clostridioformis in 2019 [29], is hardly dissociable from two other bacteria: C. bolteae and C. hathewayi, all found in human feces [30]. These 3 species are therefore usually considered as the Clostridium clostridioforme group. A higher abundance of this group in the feces has been linked to the onset of autism spectrum disorders (ASD) in children [31,32]. In rodents, PS has also been associated to an increase of ASD-like behaviours in adulthood [33-35] but, in the absence of microbiota analysis, a direct link between PS, Clostridium clostridioforme and ASD is still unknown. More studies are needed to decipher the implication of Clostridium clostridioforme in IBS but its correlation with behavioral disorders is indicative for a role in psychological comorbidities such as anxiety and depression.

The decreased abundance of the Lactobacillus genera seems to be a universal response to chronic stress as this observation has been made in various chronic stress models, applied either in adulthood or in early life, and in different species [24, 27, 36, 37]. In humans, studies looking at the correlation between stress in pregnancy and the baby's gut microbiota have also highlighted that infants of stressed mothers present a decreased abundance of Lactobacillus in their feces [38]. However, how chronic stress impacts the Lactobacillus population and its duration remain to be determined. Given that we previously showed analgesic properties of C12AsnGABA, a lipopeptide containing GABA in a model of visceral pain [14], the inverse correlation between L. murinus animalis abundance and visceral sensitivity, led us to hypothesize that this bacterium was implicated in the maintenance of the normosensitivity by producing analgesic molecules. L. murinus IRSD_2020, in the presence of GABA, produced GABA-containing lipopeptides, such as C16PheGABA, C12AsnGABA, C16GluGABA, C14AsnGABA, C12IleGABA, C14IleGABA and C12AlaGABA highlighting the redundancy to produce lipopeptides linked to GABA.

GABA production is widely distributed in all three life kingdoms: animals, plants and bacteria. In bacteria, the most studied producers belong to the lactic acid producing bacteria, a group that includes Lactobacilli [39,40]. GABA is synthetized by a pyridocal-5′-phosphate (PLP)-dependent enzyme glutamate decarboxylase (GAD; EC 4.1.1.15) by irreversible □-decarboxylation of L-glutamate and consumption of one cytoplasmic proton [41]. These enzymes are encoded by gadB and gadC genes [42]. L. murinus animalis does not possess the enzymes required to produce GABA. Nevertheless, when cultivated in a GABA enriched medium, this species forms lipopeptides linked to GABA. In vivo GABA could be provided to L. murinus animalis by two other hardly differentiable Lactobacillus species that we isolated from the mucus of control but not from PS mice, L. reuteri and the L. johnsonii gasseri cluster. In the literature, these two latter species tend to form dual-species biofilm in the gut, and are often clustering with L. murinus/animalis [43]. L. reuteri possesses the GABA production machinery [43], and the close interactions between these species in the gut could be one of the mechanisms through which L. murinus animalis acquires the GABA to produce lipopeptides containing GABA. Interestingly, we quantified a decrease of C16LeuGABA concentration in the feces of patients with IBS compared to healthy volunteers. C16LeuGABA was not quantifiable in L. murinus animalis or in mouse colon meaning that in human, this lipopeptide could be produced by other species of lactobacillus or even other species of bacteria. The diversity in lipopeptides composition could be dependent on the culture conditions or on the diet as the leucine is an essential fatty acid, which cannot be produced by the organism. In addition, bacterial environment could also have an impact on lipopeptides synthesis as, for the example, the size of fatty acids in bacteria is dependent on culture conditions and temperature [44,45]. A prospective clinical study unifying microbiota analyses and lipopeptides quantification is needed to identify bacteria implicated in the production of C16LeuGABA in the human gut microbiota. As this GABA-lipopeptide decreased capsaicin- and GPCR agonists-induced neuronal activation, the contribution of GABA-lipopeptides to visceral pain could be relevant in patients. Quantification of GABA-lipopeptides in patients would allow to identify patients which could be treated directly by the lipopeptide or by a probiotic supplemented with GABA.

GABA analogues such as gabapentin or pregabalin decrease abdominal pain in patients with IBS, but their serious side effects (hepatotoxicity and neurotoxicity) prevent their chronic use [46-48]. The analgesic properties of GABA lipopeptides, as demonstrated here for C14AsnGABA and C16LeuGABA, show that they could represent a novel therapeutic track to explore in patients with IBS. Our results show that the production of lipopeptides-GABA by commensal bacteria could be one of the mechanisms of communication between the host and its gut microbiota implicated in the upkeep of intestinal homeostasis.

### Example 3

We assessed the efficacy of C12AsnGABA, C14AsnGABA and C16LeuGABA against capsaicin in primary culture of sensory neurons.

### Methods of Calcium Imaging of Sensory Neurons

Mouse dorsal root ganglia were dissociated as previously described[N. Cenac et al., «Potentiation of TRPV4 signaling by histamine and serotonin: an important mechanism for visceral hypersensitivity», Gut, vol. 59, no 4, p. 481-488, 2010, doi: 10.1136/gut.2009.192567]. After 48 h of culture, cells were incubated with HBSS containing 20 mM HEPES, 1 mM fluo-4 acetoxymethyl (AM) and 20% pluronic F-127 for 30 min at 37° C. plus 30 min in the dark at RT. The plates were then washed with HBSS and 140 μl of HBSS were added to each well. Neurons were pre-treated with different concentration (0.1, 1, and 10 μM) of C12asnGABA, C14AsnGABA or C16LeuGABA, or vehicle (HBSS/MeOH 0.06%) for 5 min and then were stimulated with either capsaicin or the mix of GPCR agonists. Live cell imaging of calcium was carried out on an automated inverted microscope (Axio Observer, Zeiss, SAS France) with a ×10 objective (NA 0.45). Images were acquired using the Zen imaging software (3.1 Blue Edition) and a kinetic of 85 recordings (one per second) was performed. The first ten images were used to determine the baseline and, from 10 to 60 sec, neurons were exposed to either a mix of GPCR agonists (histamine, bradykinin, serotonin, 10 μM), capsaicin (500 nM) or vehicle (HBSS). After 60 seconds, neurons were treated with KCl (50 mM) in order to discriminate neurons from glial cells. The ImageJ software was used to perform the analysis of calcium flux.

### Results

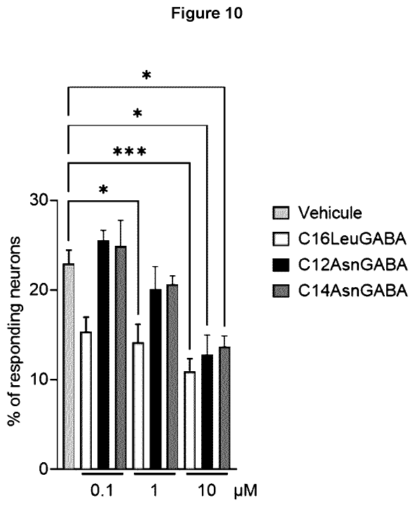

Primary cultures of mouse dorsal root ganglia (DRG) neurons, activated by an agonist of the receptor calcium channel TRPV1 (capsaicin), were treated with C12AsnGABA, C14AsnGABA or C16LeuGABA. Neurons exposure to capsaicin (500 nM) induced an increase in calcium flux as shown by the higher % of responding neurons (FIG. 10). The calcium flux increase induced by both nociceptive stimuli was prevented by C12AsnGABA, C14AsnGABA and C16LeuGABA pretreatment in a dose-dependent manner (FIG. 10). The C12AsnGABA and C14AsnGABA significantly decreased neuronal activation by capsaicin at 10 μM, in contrast, C16LeuGABA significantly decreased neuronal activation at 1 and 10 μM. In conclusion, C16LeuGABA was more potent than C12AsnGABA and C14AsnGABA.

### Example 4

We quantified the concentration of C12AlaGABA, C14:1AlaGABA, C12ValGABA, C12LeuGABA, C14GABA, C12IleGABA, C14IleGABA, C16LeuGABA, and C16PheGABA in human urine.

### Bacterial Lipopeptide Quantification

To quantify bacterial lipopeptides, mass-spectrometry method has been adapted from the C12AsnGABA quantification method previously described [T. Perez-Berezo et al., «Identification of an analgesic lipopeptide produced by the probiotic Escherichia coli strain Nissle», Nat. Commun., vol. 8, no 1, December 2017, doi: 10.1038/s41467-017-01403-9.] following our previous manuscript on the mass-spectrometry analyses of aminolipids and lipopeptides [A. Hueber et al., «Identification of bacterial lipo-amino acids: origin of regenerated fatty acid carboxylate from dissociation of lipo-glutamate anion», Amino Acids, vol. 54, no 2, p. 241-250, févr. 2022, doi: 10.1007/s00726-021-03109-1.], [A. Hueber et al., «Discovery and quantification of lipoamino acids in bacteria», Anal. Chim. Acta, vol. 1193, p. 339316, February 2022, doi: 10.1016/j.aca.2021.339316.]. For the extraction, 500 μL of Tris buffer (pH=9), 1 mL of MeOH and 5 μL of IS*C16AsnGABA were added to 300 μL of human urine. Samples were centrifuged at 1016×g for 15 min (4° C.) and the supernatants submitted to SPE using HLB plates (HLB, 30 mg, Waters). Plates were conditioned with 750 μL ethyl acetate, 750 μL MeOH and 750 μL H₂O:MeOH (90:10; v/v). Samples were loaded at a flow rate of one drop per 2 s and SPE plates washed using 1 mL H₂O:MeOH (90:10; v/v) followed by 1 mL heptane. Lipopeptides were eluted using 1 mL AcN, 1 mL MeOH and 1 mL AcOEt. Eluent were carefully removed from the plate, and transferred to a Pyrex tube, dried under nitrogen gas and reconstituted in 10 μL MeOH for LC-QqQ (ou LC-LRMS) analysis. Extract was stored at −20° C. before LC-MS analysis. High-performance liquid chromatography system was a Shimadzu Mikros LC system, equipped with a thermostated autosampler SIL-30AC, a rack changer II, a Nexera Mikros binary pump, a degasser on-line DGU-20A3R and an LTO-Mikros column oven. The analytical column was a CSH C18 column Waters (1×100 mm; 2.7 μm) and was maintained at 40° C. The mobile phases consisted of water:FA (99.9:0.1; v/v) (A) and acetonitrile:FA (99.9:0.1, v/v) (B). Flow rate was 0.1 mL·min-1. The multi-step gradient starts with 30% B at 0 min, 80% B at 13 min, 100% B at 13.5 min, 100% B at 16 min, and at 16.5 min 30% B until 18 min. The chromatography system was coupled online to a triple quadrupole mass spectrometer Shimadzu 8060 equipped with an ESI source in negative mode and was optimized as follow: nebulizer: 2 L·min-1; desolvation line: 250° C.; heating-block: 450° C., heating gas flow: 10 L·min-1, and no drying gas was used. Argon gas (purity, >99.9995%) was used for collision-induced dissociation (CID). For each metabolite the MRM transition was optimized on the pure standard to get the best selectivity and the best sensitivity with a qualitative and a quantitative transition and was programmed to monitor a 2 min-window (expected chromatographic retention time±1 min). The dwelltime were optimized for each compound and were set between 15 and 100 msec. Peak detection, integration, and quantitative analysis were performed with LabSolution software (Version 5.99 SP2), Shimadzu.

### Results

C16PheGABA, C12IleGABA, C14GABA, C12LeuGABA, C14IleGABA and C16LeuGABA were detected in the urine of the 8 healthy volunteers assessed. Distribution of the more to the less concentrated lipopeptides: C16LeuGABA>C14GABA>C12LeuGABA>C12IleGABA>C14IleGABA>C16PheGABA. The results show that the most concentrated lipopeptide is C16LeuGABA.

### REFERENCES

1 Enck P, Aziz Q, Barbara G, et al. Irritable bowel syndrome. Nat Rev Dis Primer 2016; 2:16014. doi:10.1038/nrdp.2016.14
2 Surdea-Blaga T. Psychosocial determinants of irritable bowel syndrome. World J Gastroenterol 2012; 18:616. doi:10.3748/wjg.v18.i7.616
3 Blanchard E B, Lackner J M, Jaccard J, et al. The role of stress in symptom exacerbation among IBS patients. J Psychosom Res 2008; 64:119-28. doi:10.1016/j.jpsychores.2007.10.010
4 Chang L. The Role of Stress on Physiologic Responses and Clinical Symptoms in Irritable Bowel Syndrome. Gastroenterology 2011; 140:761-765.e5. doi:10.1053/j.gastro.2011.01.032
5 Caparros-Gonzalez R A, Torre-Luque A de la, Romero-Gonzalez B, et al. Stress During Pregnancy and the Development of Diseases in the offspring: A Systematic-Review and Meta-Analysis. Midwifery 2021; 97:102939. doi:10.1016/j.midw.2021.102939
6 Olén O, Stephansson O, Backman A-S, et al. Pre- and perinatal stress and irritable bowel syndrome in young adults—A nationwide register-based cohort study. Neurogastroenterol Motil 2018; 30:e13436. doi:10.1111/nmo.13436
7 Low E X S, Mandhari M N K A, Herndon C C, et al. Parental, Perinatal, and Childhood Risk Factors for Development of Irritable Bowel Syndrome: A Systematic Review. J Neurogastroenterol Motil 2020; 26:437-46. doi:10.5056/jnm20109
8 Bradford K, Shih W, Videlock E J, et al. Association Between Early Adverse Life Events and Irritable Bowel Syndrome. Clin Gastroenterol Hepatol 2012; 10:385-390.e3. doi:10.1016/j.cgh.2011.12.018
9 Jašarević E, Howard C D, Morrison K, et al. The maternal vaginal microbiome partially mediates the effects of prenatal stress on offspring gut and hypothalamus. Nat Neurosci 2018; 21:1061-71. doi:10.1038/s41593-018-0182-5
10 Tap J, Derrien M, Törnblom H, et al. Identification of an Intestinal Microbiota Signature Associated With Severity of Irritable Bowel Syndrome. Gastroenterology 2017; 152:111-123.e8. doi:10.1053/j.gastro.2016.09.049
11 Su T, Liu R, Lee A, et al. Altered Intestinal Microbiota with Increased Abundance of Prevotella Is Associated with High Risk of Diarrhea-Predominant Irritable Bowel Syndrome. Gastroenterol Res Pract 2018; 2018:6961783. doi:10.1155/2018/6961783
12 Lepage P, Leclerc M C, Joossens M, et al. A metagenomic insight into our gut's microbiome. Gut 2013; 62:146-58. doi:10.1136/gutjnl-2011-301805
13 Huttenhower C, Gevers D, Knight R, et al. Structure, function and diversity of the healthy human microbiome. Nature 2012; 486:207-14. doi:10.1038/naturel 1234
14 Perez-Berezo T, Pujo J, Martin P, et al. Identification of an analgesic lipopeptide produced by the probiotic Escherichia coli strain Nissle 1917. Nat Commun 2017; 8:1314. doi:10.1038/s41467-017-01403-9
15 Hueber A, Gimbert Y, Langevin G, et al. Identification of bacterial lipo-amino acids: origin of regenerated fatty acid carboxylate from dissociation of lipo-glutamate anion. Amino Acids 2022; 54:241-50. doi:10.1007/s00726-021-03109-1
16 Hueber A, Petitfils C, Le Faouder P, et al. Discovery and quantification of lipoamino acids in bacteria. Anal Chim Acta 2022; 1193:339316. doi:10.1016/j.aca.2021.339316
17 Bautzova T, Hockley J R F, Perez-Berezo T, et al. 5-oxoETE triggers nociception in constipation-predominant irritable bowel syndrome through MAS-related G protein-coupled receptor D. Sci Signal 2018; 11:eaal2171. doi:10.1126/scisignal.aa12171
18 Cenac N, Bautzova T, Le Faouder P, et al. Quantification and Potential Functions of Endogenous Agonists of Transient Receptor Potential Channels in Patients With Irritable Bowel Syndrome. Gastroenterology 2015; 149:433-444.e7. doi:10.1053/j.gastro.2015.04.011
19 Lacy B E, Mearin F, Chang L, et al. Bowel Disorders. Gastroenterology 2016; 150:1393-1407.e5. doi:10.1053/j.gastro.2016.02.031
20 Francis C Y, Morris J, Whorwell P J. The irritable bowel severity scoring system: a simple method of monitoring irritable bowel syndrome and its progress. Aliment Pharmacol Ther 1997; 11:395-402. doi:10.1046/j.1365-2036.1997.142318000.x
21 Boeckxstaens G E, Drug V, Dumitrascu D, et al. Phenotyping of subjects for large scale studies on patients with IBS. Neurogastroenterol Motil Off J Eur Gastrointest Motil Soc 2016; 28:1134-47. doi:10.1111/nmo.12886
22 Zheng J, Wittouck S, Salvetti E, et al. A taxonomic note on the genus Lactobacillus: Description of 23 novel genera, emended description of the genus Lactobacillus Beijerinck 1901, and union of Lactobacillaceae and Leuconostocaceae. Int J Syst Evol Microbiol 2020; 70:2782-858. doi:10.1099/ijsem.0.004107
23 Park S H, Itoh K. Species-specific oligonucleotide probes for the detection and identification of Lactobacillus isolated from mouse faeces. J Appl Microbiol 2005; 99:51-7. doi:10.1111/j.1365-2672.2005.02584.x
24 Golubeva A V, Crampton S, Desbonnet L, et al. Prenatal stress-induced alterations in major physiological systems correlate with gut microbiota composition in adulthood. Psychoneuroendocrinology 2015; 60:58-74. doi:10.1016/j.psyneuen.2015.06.002
25 Wang H-J, Xu X, Xie R-H, et al. Prenatal maternal stress induces visceral hypersensitivity of adult rat offspring through activation of cystathionine-β-synthase signaling in primary sensory neurons. Mol Pain 2018; 14:174480691877740. doi:10.1177/1744806918777406
26 Bernatova I, Puzserova A, Balis P, et al. Chronic Stress Produces Persistent Increases in Plasma Corticosterone, Reductions in Brain and Cardiac Nitric Oxide Production, and Delayed Alterations in Endothelial Function in Young Prehypertensive Rats. Front Physiol 2018; 9:1179. doi:10.3389/fphys.2018.01179
27 Jasarević E, Howard C D, Misic A M, et al. Stress during pregnancy alters temporal and spatial dynamics of the maternal and offspring microbiome in a sex-specific manner. Sci Rep 2017; 7:44182. doi:10.1038/srep44182
28 van Bodegom M, Homberg J R, Henckens M J A G. Modulation of the Hypothalamic-Pituitary-Adrenal Axis by Early Life Stress Exposure. Front Cell Neurosci 2017; 11. doi:10.3389/fncel.2017.00087
29 Haas K N, Blanchard J L. Reclassification of the Clostridium clostridioforme and Clostridium sphenoides clades as Enterocloster gen. nov. and Lacrimispora gen. nov., including reclassification of 15 taxa. Int J Syst Evol Microbiol 2020; 70:23-34. doi:10.1099/ijsem.0.003698
30 Finegold S M, Song Y, Liu C, et al. Clostridium clostridioforme: a mixture of three clinically important species. Eur J Clin Microbiol Infect Dis 2005; 24:319-24. doi:10.1007/s10096-005-1334-6
31 Finegold S M, Molitoris D, Song Y, et al. Gastrointestinal Microflora Studies in Late-Onset Autism. Clin Infect Dis 2002; 35:S6-16. doi:10.1086/341914
32 Song Y, Liu C, Finegold S M. Real-Time PCR Quantitation of Clostridia in Feces of Autistic Children. Appl Environ Microbiol 2004; 70:6459-65. doi:10.1128/AEM.70.11.6459-6465.2004
33 Weinstock M. Prenatal stressors in rodents: Effects on behavior. Neurobiol Stress 2017; 6:3-13. doi:10.1016/j.ynstr.2016.08.004
34 Kinney D, Munir K, Crowley D, et al. Prenatal stress and risk for autism. Neurosci Biobehav Rev 2008; 32:1519-32. doi:10.1016/j.neubiorev.2008.06.004
35 Weinstock M. The long-term behavioral consequences of prenatal stress. Neurosci Biobehav Rev 2008; 32:1073-86. doi:10.1016/j.neubiorev.2008.03.002
36 Hantsoo L, Zemel B S. Stress gets into the belly: Early life stress and the gut microbiome. Behav Brain Res 2021; 414:113474. doi:10.1016/j.bbr.2021.113474
37 Mackos A R, Varaljay V A, Maltz R, et al. Role of the Intestinal Microbiota in Host Responses to Stressor Exposure. In: International Review of Neurobiology. Elsevier 2016. 1-19. doi:10.1016/bs.irn.2016.08.002
38 Zijlmans MAC, Korpela K, Riksen-Walraven J M, et al. Maternal prenatal stress is associated with the infant intestinal microbiota. Psychoneuroendocrinology 2015; 53:233-45. doi:10.1016/j.psyneuen.2015.01.006
39 Dhakal R, Bajpai V K, Baek K-H. Production of gaba (& #947; -aminobutyric acid) by microorganisms: a review. Braz J Microbiol 2012; 43:1230-41. doi:10.1590/S1517-83822012000400001
40 Seok J-H, Park K-B, Kim Y-H, et al. Production and Characterization of Kimchi with Enhanced Levels of γ-Aminobutyric Acid. 7.
41 Ueno H. Enzymatic and structural aspects on glutamate decarboxylase. J Mol Catal B Enzym 2000; 10:67-79. doi:10.1016/S1381-1177(00)00114-4
42 Ma D, Lu P, Yan C, et al. Structure and mechanism of a glutamate-GABA antiporter. Nature 2012; 483:632-6. doi:10.1038/nature10917
43 Lin X B, Wang T, Stothard P, et al. The evolution of ecological facilitation within mixed-species biofilms in the mouse gastrointestinal tract. ISME J 2018; 12:2770-84. doi:10.1038/s41396-018-0211-0
44 Núñez-Cardona M T. Influence of culture conditions on the fatty acid composition of green and purple photosynthetic sulphure bacteria. IntechOpen 2014. doi:10.5772/57389
45 Suutari M, Laakso S. Microbial fatty acids and thermal adaptation. Crit Rev Microbiol 1994; 20:285-328. doi:10.3109/10408419409113560
46 Cui Y, Miao K, Niyaphorn S, et al. Production of Gamma-Aminobutyric Acid from Lactic Acid Bacteria: A Systematic Review. Int J Mol Sci 2020; 21:995. doi:10.3390/ijms21030995
47 Zhang M-M, Liu S-B, Chen T, et al. Effects of NB001 and gabapentin on irritable bowel syndrome-induced behavioral anxiety and spontaneous pain. Mol Brain 2014; 7:47. doi:10.1186/1756-6606-7-47
48 Saito Y A, Almazar A E, Tilkes K E, et al. Randomised clinical trial: pregabalin vs placebo for irritable bowel syndrome. Aliment Pharmacol Ther 2019; 49:389-97. doi:10.1111/apt.15077

1. A compound of Formula (I): 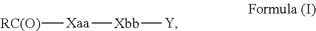
wherein
R is a C5-C19 linear or branched hydrocarbon chain selected from the group consisting of alkyl, alkene, and alkyne,
Xaa is leucine, phenylalanine, isoleucine or alanine,
Xbb is HNCH₂CH₂CH₂CO,
Y is —OH or NH₂,
and wherein Xbb is linked to Xaa through its amine functional group and wherein the RC(O) group is at the N terminal side and Y is a C terminal side,
or a pharmaceutical acceptable salt thereof.

2. The compound according to claim 1, wherein R is a C5-C19 alkyl.

3. The compound according to any of claim 1, wherein R is a C12 alkyl or a C14 alkyl or a C16 alkyl.

4. The compound according to claim 1, wherein Y is —OH—.

5. The compound according to claim 1, wherein the compound is C15 C(O)-Phe-gamma-aminobutyric acid.

6. The compound according to claim 1, wherein the compound is C11 C(O)-Ile-gamma-aminobutyric acid.

7. The compound according to claim 1, wherein the compound is C13 C(O)-Ile-gamma-aminobutyric acid.

8. The compound according to claim 1, wherein the compound is C11 C(O)-Ala-gamma-aminobutyric acid.

9. The compound according to claim 1, wherein the compound is C15 C(O)-Leu-gamma-aminobutyric acid.

10. A method for treating a subject in need thereof, the method comprising administering to the subject a compound according to claim 1, wherein the compound is used as a drug.

11. A method for treating a pain disorder in a subject in need thereof, the method comprising administering to the subject a therapeutically effective amount of a compound according to claim 1.

12. (canceled)

13. (canceled)

14. A pharmaceutical composition, comprising a compound according to claim 1, and one or more pharmaceutically acceptable excipients.

15. A method to obtain the compound according to claim 1, comprising synthesizing the compound using a peptide solid phase synthesis technique or a liquid phase synthesis technique.

16. The method according to claim 11, wherein the pain disorder is a visceral pain disorder or a somatic pain disorder.

17. The method according to claim 16, wherein the visceral pain disorder is Irritable Bowel Syndrome (IBS) or Inflammatory Bowel Disease (IBD).

18. The compound for use according to claim 16, wherein the visceral pain disorder is resulting from a urogenital disorder selected from the group consisting of a bladder neoplasm, a chronic urinary tract infection, interstitial cystitis, radiation cystitis, recurrent cystitis, recurrent urethritis, urolithiasis, uninhibited bladder contractions, urethral diverticulum, chronic urethral syndrome, a urethral carbuncle and a urethral stricture.
